(19) United States
(12) Patent Application Publication
(10) Pub. No.: US 2026/0287584 A1
(43) Pub. Date: Sep. 24, 2026

(54) IVA TEST UNIT, A TEST SET COMPRISING THE IVA TEST UNIT AND A METHOD OF PRODUCING THE SAME

(71) Applicant: Roche Diagnostics Operations, Inc., Indianapolis, IN (US)

(72) Inventors: Martin Blum, Edingen-Neckarhausen (DE); Yilmaz Isgoeren, Ludwigshafen (DE); Sascha Meyer Dos Santos, Frankfurt a. Main (DE)

(21) Appl. No.: 19/642,591

(22) Filed: Apr. 8, 2026

Related U.S. Application Data
(63) Continuation of application No. PCT/EP2024/078092, filed on Oct. 7, 2024.

(30) Foreign Application Priority Data
Oct. 10, 2023 (EP) 23 202 563.5

(51) Int. Cl. G01N 33/543

(52) U.S. Cl. CPC G01N 33/54388

(57) ABSTRACT

An IVA test unit has a test strip for detecting an analyte. The test strip has at least an application function, a test reaction function and a test detection function. A housing is provided for storing the test strip and the housing has an insert tray and an insert hole for inserting the test strip into the insert tray. The housing is formed as one single piece.

### RELATED APPLICATIONS

This application is a continuation of PCT/EP2024/078092, filed Oct. 7, 2024, which claims priority to EP 23 202 563.5, filed Oct. 10, 2023, the entire disclosures of both of which are hereby incorporated herein by reference.

### BACKGROUND

This disclosure relates to an In Vitro Assay (IVA) test unit, specifically a lateral flow assay (LFA) test unit and a method of producing the same in the field of In Vitro Diagnostics (IVD) as well as a test set comprising the IVA test unit.

Conventional LFAs, such as LFA-strips or dip stick tests are often used in diagnostics applications. Test membranes which may be used in such tests may specifically include, without limitation, a nitrocellulose membrane. Specific lines are typically applied to the membrane containing specific chemical capturing reagents or compounds, that are designed to react and bind with predetermined analytes, specifically biological markers which may be present in test sample liquids such as, but not limited to, sputum, urine, blood, water, liquefied food samples and surface swabbed samples. Specific examples of LFAs are rapid antigen tests for detecting the presence of analytes, such as virus fragments of infectious diseases in a biological sample. In general, LFA test units are often used in the form of test strips, such as, for example, pregnancy test strips, drug test strips, influenza and/or Covid 19 tests and/or allergen rapid tests.

When a sample liquid is applied onto an application region of an LFA test strip, the sample liquid is drawn by capillary forces along a longitudinal axis of the membrane (also called membrane analyte assay strip). The presence of predetermined analytes is typically indicated when enzymes, target proteins and/or bio-markers corresponding to these analytes migrate along the membrane and interact with the chemical capturing reagents in the stripes of the membrane, thereby producing measurable and detectable signals/changes the analyte assay test zone(s). The resulting detectable and measurable signals at striped zones may, without limitation, be caused by binding with colloidal gold, charcoal beads, latex dyed beads, magnetic or paramagnetic beads and/or agents or particles capable of emitting fluorescence, auto-fluorescence, luminescence, phosphorescence, or chemiluminescence.

Test sets/test kits which are based on such test units, specifically LFA test units, comprise several individual elements, each having a different function, and the production, as well as the assembly thereof is complex, difficult and/or time-consuming. Therefore, an increase of the production yield requires high efforts in terms of machine performance. Moreover, it requires multiple assembly steps to provide an assembled test set that is packaged and ready to be shipped. Often, the volume and the shape of the package do not allow an easy stacking of multiple packages and require a lot of space.

In FIG. 1, elements of a typical LFA-set 200 are shown, namely an LFA-rapid antigen test set for detecting the presence of Covid 19 in a biological sample. The comprised LFA-rapid antigen test usually corresponds to a single-use test, which means that it is disposed after the test is performed, therefore the test set is considered a disposable. The LFA-rapid antigen test comprises a test strip, specifically an LFA-core strip 202 and a plastic housing 201, which consists of at least two pieces, namely an upper piece (a cover piece) and a lower piece (a support). Further, the LFA-set 200 comprises a swab for collecting a biological sample (not shown), a drying agent/desiccant 204 and a water-vapor-impermeable aluminum pouch 203 into which the LFA-rapid antigen test, the swab and the drying agent 204 are inserted, packed and sealed. The LFA-rapid antigen test is typically assembled by means of a pick-and-place procedure, by which the LFA-core strip 202 is positioned on the lower piece of the plastic housing 201, being followed by a step of assembling and clipping the upper piece of the plastic housing 201 onto the lower piece to form an LFA-unit (LFA-rapid antigen test in the present case). The production may be time-intensive and in some cases inefficient. Also, the shipping of such LFA-rapid antigen test sets may often be inefficient. Moreover, the costs of production and/or shipping of such LFA-rapid antigen test sets may be high.

### SUMMARY

It is therefore desirable to provide a simplified, fast and/or efficient method of producing an IVA test unit. A method of producing a high number of IVA test units in a short time is specifically needed in a pandemic situation. Such a dynamic situation often requires the possibility of a simple and/or quick up-and/or down scaling of the production rate to account for an increase and/or decrease of infections. Providers can therefore increase the degree of agility and quickly react to a changing pandemic situation. A pandemic situation also often requires that a high number of test sets/kits can be efficiently and/or quickly shipped to the point-of-care and/or the place of pandemic outbreak. Therefore, it is also desirable to provide an improved and/or more compact IVA test unit and/or test set specifically in terms of logistics, appearance and/or function.

According to an aspect, which may be considered the first aspect of the present disclosure, an IVA test unit comprises a test strip for detecting an analyte in a sample liquid, wherein the test strip comprises at least an application function, a test reaction function and a test detection function; and wherein the IVA test unit comprises a housing for storing the test strip, and wherein the housing comprises an insert tray and an insert hole for inserting the test strip into the insert tray, characterized in that the housing is formed as one single piece. In other words, the IVA test unit comprises a single-piece housing.

The IVA test unit may, without limitation, be part of a test set. The IVA test unit according to the first aspect has a simplified and compact structure since the housing is formed from a single piece and hence the number of elements can be reduced, which makes it easier to assemble the IVA test unit. Particularly, the production steps can be simplified and/or the number of production steps can be reduced. It is not required to assemble two or more elements of the housing, for example, during a complex pick-and-place procedure. Further, it is not required to bond elements of the housing as the housing only consists of one single element. The test strip can be easily positioned inside the housing by inserting (specifically pushing) the test strip through the insert hole, substantially corresponding to a slit, into the insert tray, which reduces the production complexity, the time needed for the production and/or the costs. It is hence simple and cheap to adjust and/or increase the production volume of the IVA test units. In a pandemic situation, the production rate can therefore be easily up scaled (increased) and/or downscaled (decreased) to account for increasing and/or decreasing infection rates and therefore, the producer and/or supplier of test sets and/or test kits can easily and quickly react to sudden changes in a pandemic situation. On the one hand, the possibility of a quick and efficient up scaling may allow to quickly provide many test sets if the infection rates shoot up. On the other hand, the possibility of a quick and efficient down scaling may allow to quickly reacting to a sudden drop in infection rates, possibly due to the establishment of a herd immunity and avoids an overproduction of test sets, which are not needed any longer.

The single-piece housing does further not involve the risk that pieces of the housing disassemble since it only consists of one piece. Further, a single-piece housing does not comprise a coupling mechanism for coupling together elements of the housing and therefore no coupling mechanism can be damaged and cause erroneous disassembly.

Further, the single-piece housing provides a high stability and therefore a high protection of the test strip. Specifically, the single-piece housing does not easily twist.

Besides the improvements in terms of efficiency and function, the IVA test unit may also appear to a user as a high quality product. The IVA test unit may also appear as being improved in terms of esthetics.

The housing is formed as one single piece. Specifically, the housing may be formed as one single piece by means of injection molding and/or additive manufacturing. All regions/portions of the housing which are produced together by injection molding and/or additive manufacturing can be considered as being a single piece. A single piece is characterized in that all elements, regions and portions of the single piece cannot be reversibly disassembled from each other and reassembled to obtain again the single piece without destruction. The elements of a single piece housing would lose their functionalities in an attempt to disassemble them from each other.

The IVA test unit may, without limitation, correspond to or comprise an LFA-unit that comprises a test strip, which corresponds to an LFA-corestrip. The LFA-corestrip may, without limitation, comprise individual dedicated zones for specific functions. Such functions may, without limitation, correspond to an application function, a test reaction function and a test detection function. A zone comprising a sample pad typically has the application function. A zone comprising a conjugate pad typically has the test reaction function where the binding between a molecule and a conjugate takes place. A zone comprising a membrane may serve as a test detection zone and may comprise a test line and a control line to detect the analyte of interest if present in the sample. Other test strips may be used in addition or instead. Therefore, the IVA test unit may comprise another type of IVA test unit, such as a urine test, for example, and without limitation. Such alternative tests may, for example, have one zone and/or one pad, which comprises all three functions in one position, namely the application function, the test reaction function and the test detection function. Test membranes which may be used in such tests may include, without limitation, nitrocellulose, silicone, plastic, glass, glass fiber, polyvinylidene fluoride, nylon that is optionally charge modified, polyethersulfone, aluminum foil, paper, and/or other porous cellulose and rayon compounds being hydrophilic or hydrophobic. The membrane may further comprise an immobilized binder for the specific analyte. Such a binder may be any molecule or bio-molecule with which the biomolecular analyte of interest interacts so as to result in a binder-analyte/ligand complex that is immobilized onto the membrane. Examples of such binders may be an antibody, antigen, protein, enzyme or part thereof, substrate or part thereof, peptide, DNA, or RNA.

The insert hole may, without limitation, be located on a short side of the housing, wherein the housing has a longitudinal axis along a length of the housing which relates to a side that is longer than the short side. The insert hole may, without limitation, be located centered on a surface of the short side and/or with respect to the central longitudinal axis or may be located off-center. Alternatively, (but not preferably) the insert hole may be provided at or on the long side of the housing.

The analyte may comprise at least one of the following, without limitation: a virus or a fragment of a virus, such as a spike protein, a compound of a bacteria, a hormone, a molecule produced by a body, a molecule produced by a foreign organism. The IVA test unit may comprise at least one of the following, without limitation: a pregnancy test strip, a drug test strip, an influenza and/or Covid 19 test and/or an allergen rapid test. The IVA test unit may be specific to one analyte or to several analytes in a combined test.

The housing may comprise a first wall, such as an upper wall, and a second wall, such as a lower wall, that is adjacent the first wall and wherein the first wall and the second wall form and/or define the insert tray at least partially as a gap between the first wall and the second wall. The first wall and the second wall are formed as a single piece and may, in some cases, be therefore considered as a single wall. In such a case, the first wall may be considered a first portion of the wall and the second wall may be considered a second portion of the same wall.

It is not required, that the first and/or the second walls are “closed” walls, i.e., fully surrounding the insert tray. It may specifically be that one or both walls comprise(s) one or more recess(es) and/or through holes. For example, the first wall may comprise an application recess and/or a recess in the control area. In that case, both walls, specifically the residual portions of both walls may still define the insert tray.

The housing may generally be understood as an element that has two opposing walls and/or sides between which the test strip can be positioned at least partially. In other words, the housing may have an upper portion, which may correspond to the first wall that faces a lower portion, which may correspond to the second wall of the housing thereby forming a gap between both portions for inserting the test strip. The test strip may hence be at least partially surrounded by the housing. In general, the housing may comprise three to six walls if a box-like housing is provided. The walls of the housing are formed as a single-piece. In many cases, the housing comprises substantially six walls, specifically six walls that are arranged perpendicularly to each other. If all walls on all sides are considered one single wall and/or envelope, each side may be considered a portion of the wall and/or envelope. The housing may, in specific embodiments, enclose the test strip to the extent of approximately 51-99%, preferably of approximately 65-95% and even more preferably of approximately 70-90%, such that most of the test strip is surrounded by the housing, wherein only a portion of the test strip at an application recess of the housing, a control area of the housing and/or the insert hole may not be covered, enclosed and/or surrounded by the housing. A very high degree of coverage, such as approx. 90% to 98% is achieved if only the application recess leaves an open space and the control area is closed, for example, in a transparent housing which allows a user to read the test result. The housing should not be understood as an element that substantially forms a base plate onto which the test strip is positioned and onto which a cover plate is positioned to enclose and/or secure the test strip. In that case, the base plate and the cover plate would form a housing and would hence not correspond to a single piece as required by this disclosure.

The housing may further be understood as a protective element of the test strip, wherein the housing has a higher degree in stability than the test strip to mechanically support and/or protect the test strip. As already described before, the housing should be understood as an element with a first side and an opposing second side of a single piece forming the gap for the insert tray. If one side is recessed, a cover element may be used to cover the recess but the cover element is not considered as part of the housing herein since the housing is a single piece. In general, other elements, such as a cap, an insert plug, a cover plate, a foil and/or a film may not be considered as being elements of the housing herein, if they are not explicitly formed with the housing as a single piece. However, specifically a cap, an insert plug and/or a cover plate may be formed as a single piece with the residual portions of the housing being connected with the housing by flexible, bendable and/or thin bridge element and/or a band and/or another flexible connection element which is also formed as a single piece with the main portion of the housing and the cap, the insert plug and/or the cover plate. In other words, such elements (a cap, an insert plug and/or a cover plate) may be integrally formed as a single-piece with the entire housing and no assembly step is therefore required for their attachment to the housing. In that case, the cap, the insert plug and/or the cover plate may be considered as being a portion of the housing. The flexible bridge element and/or connection element may allow to remove the cap, the insert plug and/or the cover plate from a recess and to open the housing while at the same time the connection is maintained and the cap, the insert plug and/or the cover plate do not get lost.

Typically, in conventional test sets, a housing of a test strip comprises at least two elements, such as a bottom element and a top element with a coupling mechanism, wherein the at least two elements may be sandwiched and coupled together by the coupling mechanism in a pick-and-place procedure, after a test strip is positioned on or in one of the elements. The housing produced by this conventional method allows fixing and/or securing a test strip inside the housing, as the housing is assembled after the test strip is placed on or inside one of the at least two elements. The IVA test unit may allow securing the test strip in easy ways, as outlined for different embodiments below.

The insert tray may correspond to an internal volume of the housing. The insert tray may provide a volume for housing the test strip that is approximately 1.2 to 20 times or more larger than the volume of the test strip. Specifically, the height of the housing may be approximately 1.2 to 20 times or more larger than the height of the test strip. The same applies for the width. It is advantageous to provide a somewhat broader and/or higher insert hole and/or insert tray to make insertion of the test strip into the insert tray easy. The insert tray may, without limitation, have a tapered insert hole (and/or conically shaped), which makes it even easier and/or more reliable to insert the test strip while the risk is reduced that the test strip falls out of the insert tray. This may be advantageous specifically if a user manually performs the insertion but also is a robot or machine performs the insertion. Alternatively, in some embodiments, the insert tray and/or the insert hole is not tapered and/or conically shaped.

The insert tray may have a surface that corresponds approximately to the area (length×width) of the test strip (or is slightly larger).

The insert tray may have a lower surface that is positioned lower than the insert hole. In other words, the insert tray is set back and/or lower further into the lower wall of the housing with respect to the lowest point of the insert hole or at least the lower surface defined by the insert hole. Again, in other words, the lower wall of the housing which defines the lower surface of the insert tray may be recessed inside the interior volume to receive the test strip in a position that is lower than the insert hole. In that way, the test strip may be inserted into the insert tray via the insert hole and falls down into the insert tray driven by gravity such that it is supported by the lower surface of the insert tray. Without limitation, the recess, i.e., the distance between the lowest point of the insert hole and the lower surface of the insert tray, may be approximately 0.1 cm to 1 cm, specifically approximately 0.2 cm to 0.7 cm and preferably approximately 0.3 cm to 0.5 cm. This may help securing the test strip inside the insert tray such that it does not fall out easily.

Alternatively or in addition, the insert hole, which substantially corresponds to a slit, may be tilted with respect to a plane that is parallel to the substantially parallel (horizontal) planes defined by the upper and the lower walls of the housing. The test strip and/or the housing may therefore be slightly tilted with respect to each other (and/or with respect to the horizontal plane) when inserting the test strip into the insert tray. Without limitation, the slit axis of the insert hole may be tilted with respect to the plane that is parallel to the substantially parallel (horizontal) planes defined by the upper and the lower walls of the housing by an angle α of approximately 3° to 45°, specifically by approximately 5° to 30°, and preferably by approximately 7° to 15°. As a result, the angle between the height axis of the housing (which is perpendicular to the substantially parallel and horizontal planes defined by the upper and the lower walls of the housing) and the slit axis of the insert hole may be slightly off 90° (90°+/−approximately 3° to 45°, specifically by approximately 90°+/−5° to 30°, and preferably by approximately 90°+/−7° to 15°). A tilted insert hole allows securing the test strip inside the insert tray in a simple way as the probability of falling out of the insert tray via the tilted insert hole is very low since there is no surface that supports the test strip in that angle and therefore, it does not easily fall out.

Alternatively or in addition, the insert hole may be only slightly larger than the test strip in its cross section such that it is not easy for the test strip to meet the exact position of the insert hole, thereby reducing the risk of falling out. The insert hole may be tapered towards the insert tray. The insert hole, i.e., a tight slit, may specifically be only slightly larger than the test strip on the inner side of the insert hole, i.e., the side that is closest to the insert tray. Without limitation, the cross section area of the insert hole may be larger than the cross section area of the test strip by the factor of approximately 1.05 to 5, specifically by the factor of approximately 1.1 to 2 and preferably by the factor of approximately 1.2 to 1.7. If the insert hole is tapered towards the insert tray, it allows an easy insertion and the insert tray guides the test strip with its tapered form towards the insert tray. However, once the test strip is inserted, it does not easily fall out via the insert hole, as the insert hole corresponds to a tight slit which may be positioned higher than the insert tray, which may be tilted with respect to the upper and lower walls of the housing and/or which may be only slightly larger than the test strip itself.

Alternatively or in addition, the insert hole may be (slightly) curved and inserting the test strip may therefore require a (slight) bending of the test strip to meet the shape of the opening of the insert hole. The bending may be performed and/or supported by an additional support that may be placed above and/or underneath the test strip and that forces the test strip into the required shape to be inserted via the curved insert hole. Alternatively or in addition, the insert hole may be tapered from the outside surface towards the inside surface from a less curved or substantially uncurved slit on the outside towards a curved slit on the inside and in such a way that the test strip is forced into the (slightly) bent and/or curved shape by the insertion process, specifically by a pushing movement of the test strip into the insert tray. As most test strips are resilient in shape and turn back into their flat shape once being inserted, they are safe from falling out of the insert tray, as they do not spontaneously adopt the curved shape.

Alternatively or in addition, the insert hole may comprise a flexible lip on the inside of the insert tray which bends towards the insert tray when inserting the test strip and covers or even seals the insert hole from inside the insert tray. The flexible lip corresponds to a valve in its function. The test strip is then secured by the flexible lip inside the insert tray and does not fall out via the insert hole since the flexible lip is configured to only bend in one direction. The flexible lip can also protect the insert tray from contaminations, water and/or water vapor.

All these features may be realized in a relatively simple way by means of the said manufacturing methods (injection molding and/or additive manufacturing) and may therefore effectively help securing the test strip inside the housing.

Therefore, resuming the above, the IVA test unit may comprise at least one of the following features to reduce the risk of the test strip falling out from the insert tray:
a lower surface of the insert tray (i.e., an (upper) surface of the above described second (lower) wall on the inside of the housing adjacent the (lower) surface of the first wall on the inside of the housing) may be positioned lower than the lowest point of the inner side of the insert hole, specifically the lower surface of the insert tray is approximately 0.1 cm to 1 cm, specifically approximately 0.2 cm to 0.7 cm and preferably approximately 0.3 cm to 0.5 cm deep with respect to the lowest point of the inner side of the insert hole;
the insert hole may be tapered from the outer side of the insert hole towards the inner side of the insert hole, specifically such that the cross section area defined by the outer side of the insert hole is larger than the cross section area defined by the inner side of the insert hole and the side walls of the insert hole taper from the outer side of the insert hole to the inner side of the insert hole;
the insert hole, specifically the inner side of the insert hole may be tilted by the angle α with respect to a plane parallel to the first and the second walls of the housing, specifically the insert hole may define a slit with a slit axis, i.e., the length axis of the insert hole specifically on the inner side, and the slit axis may be tilted with respect to the plane parallel to the first and the second walls of the housing and form an angle with the height axis of the insert hole that is off 90°, namely 90°+/−α;
the inner side of the insert hole may be formed in a bent shape and/or may be configured to bent the test strip when inserting the test strip into the insert tray, specifically the outer side of the insert hole may be tapered towards the inner side of the insert hole such that the test strip is brought further and further into a bent shape that fits the inner side shape of the insert hole upon inserting the test strip;
the IVA test unit may comprise a flexible and/or resilient flap that is configured to cover the insert hole from the inner side of the insert hole, to bend inwards when inserting the test strip and flip back and cover the insert hole when the test strip is fully inserted and prevent the test strip from falling out via the insert hole.

The housing may, without limitation, be at least partially transparent. The housing may specifically be fully transparent. If the housing is at least partially transparent, specifically in the control area, the user may check the state of the test strip inside the housing and/or read the test result through one transparent wall of the walls. The housing may then have the function of enclosing the test strip while at the same time providing a view onto the test strip, specifically onto the test detection zone such that the user may read the test result from a test line of the test strip.

The housing may, without limitation, comprise an application recess for allowing the application of a sample liquid onto the test strip. Alternatively or in addition, the housing may, without limitation, comprise a control area for reading out a test result from the test strip.

The application recess may allow a user to apply a sample liquid onto the test strip through the application recess. The application recess may, without limitation, correspond to a through hole to reach the insert tray with tip of a pipette and/or syringe and/or applicator to introduce a sample liquid onto a test strip. The user may, without limitation, use a syringe, a pipette, an applicator, a vial, a tube or the same to apply the sample liquid. The syringe, pipette, applicator, tube and/or vial may, without limitation, be provided together with the test set. The application recess may be covered, for example, by means of a membrane and/or a cover plate to seal the inside of the housing. The membrane may then be punctured by means of a needle of a syringe or a pipette before applying the sample liquid. A cover plate may be simply removed to reach the test strip.

The control area may, without limitation, correspond, in one embodiment, to a through hole, which allows the user to read the test result from the test line of the test strip. In this case, the housing does not completely (100%) surround the test strip in the control area. The width of the housing may, without limitation, be approximately 1,5 to 20 times larger than the width of the through hole of the control area. The length of the housing may, without limitation, be approximately 1,3 to 15 times larger than the length of the through hole of the control area.

Alternatively or in addition, the control area may, without limitation, correspond to and/or comprise a transparent area of the housing, which allows the user to read the test result from the test line of the test strip. Alternatively or in addition, the control area may, without limitation, correspond to and/or comprise an area that comprises at least one sensor, at least one access and/or recess for a sensor and/or at least one receiving position for a sensor. The sensor may specifically comprise an optical sensor. The sensor may, without limitation, comprise a camera. The sensor may record and/or read out a test result from the test strip. Otherwise, if optically accessible to a user, the user may read out a test result from the test strip. Typically, in a simple disposable which is used as a home testing unit (tested by private users at home) and/or a point-of-care unit, it is useful that the test result can be visually read by the user him/herself or another person that is not required to be a professional health care worker. However, alternatively or in addition, in a laboratory, an IVA test unit may, without limitation, be read out in an automated manner, for example, by a machine. This allows to automatically read out a manifold of test results and/or to automatically enter the test result into a data base. In that case, the machine for reading out the result may, without limitation, be used over a long period while the IVA test units are used as disposables.

The IVA test unit may, without limitation, comprise one or more of the following closure elements: an insert plug configured to be plugged into the insert hole to close the insert hole and/or secure the test strip from falling out of the insert tray; a cap configured to be clipped onto the housing in and/or over the insert hole.

A closure element, such as the insert plug and/or the cap is configured to open and/or close the insert hole for a single time and/or repeatedly. A closure element may seal the insert tray. In that case, there is no exchange of air and/or water vapor between the outside and the insert tray. An insert plug may be fitted to the insert hole and may be plugged into the insert hole, such that it reaches into the insert tray. A cap may instead substantially cover the insert hole without reaching into the insert tray. A combination of both possible closure elements may be realized by a cap having an insert plug such that the insert hole is covered and sealed at a high reliability.

The insert plug and/or the cap may, without limitation, be formed integrally with the housing, i.e., as a single piece with the housing. In that case, the insert plug and/or the cap is formed with a flexible, bendable and/or thin bridge element that connects the insert plug and/or the cap with the housing to form a single piece.

The closure element may alternatively comprise a foil, specifically a water-vapor-impermeable foil and/or another element that is attached to the housing, for example, glued, welded and/or laminated to the housing and covers the insert hole and/or reaches into the insert hole and/or covers another recess. In one embodiment, specifically in a single-use IVA test unit, the foil and/or foils may be removed before use and may not be configured to seal the interior of the housing once it was removed. This allows sealing the interior of the housing against water vapor, water, gases and/or contaminations with a small amount of material in a simple and/or efficient manner. In general, the interior volume of the housing may be filled with a gas, specifically an inert gas and/or a dry gas to prevent early deterioration of the test strip.

The housing may, without limitation, comprise a holding mechanism that is configured to hold one, several or all elements of the test set/kit, such as the swab and/or a vial. The vial may, without limitation, comprise, store and/or contain a liquid, such as a buffer solution in which a sample, that is collected with the swab, can be eluted. The holding mechanism may, without limitation, comprise a clamp, a clipping element and/or a recessed element and/or the like to hold and/or secure the swab and/or the vial with the buffer solution. At least one holding mechanism may be formed integrally with the housing. In other words, at least one holding mechanism may be formed as a single piece together with the housing. The holding mechanism may, without limitation, comprise a breaking point such that a user can break and/or disassemble the holding mechanism to remove the swab and/or the vial from the housing together with the holding mechanism. Alternatively or in addition, the housing may, without limitation, comprise one or more recesses and/or holes to house and/or secure the vial and/or the swab. In the latter case, the recesses may be fitted to the shape of the vial and/or the swab such that the vial and/or the swab do not exceed the shape of the housing. By coupling the vial and/or the swab onto the housing, a very compact test set can be provided. The test set can be stapled and shipping becomes very efficient. The test set may be tightly packed with a water-impermeable and specifically a water-vapor-impermeable foil and/or may be covered with a water-vapor-impermeable film. The swab and/or a head of the swab may, without limitation, be packed individually to provide clinical purity.

The IVA test unit may, without limitation, comprise at least one cover plate, specifically a transparent cover plate or a partially transparent cover plate configured to cover and/or close the control area and/or the application recess and/or other recesses. As mentioned before, The cover plate may be formed as a single piece with the residual portions of the housing being connected with the housing by a flexible, bendable and/or thin bridge element and/or a band and/or another flexible connection element which is also formed as a single piece with the main portion of the housing and the cover plate. In that case, the cover plate may be considered as belonging to the housing.

A cover plate may seal the inside volume, i.e., the insert tray in which a test strip is located while at the same time, when placed in and/or over the control area, while the transparent cover plate may in addition allow visible access for a user, such that the user can monitor the state of the test strip (used state or unused state) and/or read a result. The cover plate may in addition be covered and/or sealed by means of a water-vapor-impermeable foil.

A cover plate that is configured to cover the control area may, without limitation, be fixed, attached and/or glued to the housing such that a user cannot remove the cover plate without substantially destroying the IVA test unit. Alternatively, the transparent cover plate may be placed in and/or over the control area and may be configured for being easily and/or reversibly removed from the housing. Specifically in the latter case, it may not be required that the cover plate is transparent and it may therefore be opaque, at least partially. The cover plate may also have the function of sealing the inside volume of the housing, such as the insert tray and the test strip therein, before the IVA test unit is used. In this case, it may be possible that a foil and/or a film is used in addition or instead of a cover plate to cover and/or close the control area. The foil and/or film may be removed shortly before the IVA test unit is used.

A cover plate which is positioned in and/or over the application recess may, without limitation, be configured for being removed from the housing such that the user can reach the test strip for applying the sample liquid onto the test strip. Alternatively or in addition, the cover plate may have a recess to allow a user to reach the test strip when applying the sample liquid. The cover plate in and/or over the application recess may, without limitation, have the main function of sealing the inside volume of the housing, such as the insert tray and the test strip therein, before the IVA test unit is used. In that case, it may also be possible that a foil and/or a film instead of a cover plate is used to cover and/or close the application recess of the housing and/or a recess in the cover plate over the application recess. The foil and/or film may be removed shortly before the IVA test unit is used. In general, the foil and/or film over any recess may be glued, laminated and/or fused to the housing. If the foil and/or film is not yet removed, it allows a user to recognize that an IVA test unit is new and has not been opened and/or used already.

A cover plate (transparent or opaque) over the application recess and the cover plate over the control area, specifically a transparent cover plate, may correspond to isolated, individual and/or different cover plates, which are separate from each other. Alternatively, only one cover plate and/or foil/film is provided to cover and/or close more than one recess. For example, one single cover plate and/or one single foil may cover the control area (which may be a recess in the housing) and the application recess, at the same time.

In any case, the cover plate(s), foil(s) and/or film(s) is/are not to be understood as part and/or element of the housing. The cover plate(s), foil(s) and/or film(s) is/are typically not formed in one single piece with the housing.

In a case when the housing is produced by an additive manufacturing principle, like 3D printing, it may be possible that different materials are used for different portions of the housing and therefore, a transparent material may be used to form the control area, while other portions are produced using another material, for example, an opaque material. However, in that specific case, there is no cover plate provided. A cover plate (which is not formed as a single piece with the housing) is characterized in that it exists as a separate element that is not a portion of the housing and that the production of the IVA test unit requires to assemble the cover plate into/onto the housing. All regions and/or portions of the housing which are produced together by injection molding and/or additive manufacturing can be considered as being a single piece.

The IVA test unit may, without limitation, comprise a drying agent/desiccant as a compound of the housing. The IVA test unit may, without limitation, comprise a drying agent/desiccant as a compound of the closure element. The IVA test unit may, without limitation, comprise a drying agent/desiccant as a compound of the cover plate. The IVA test unit may, without limitation, comprise a drying agent/desiccant as an element placed in or on at least one chamber and/or recess of the housing. The IVA test unit may, without limitation, comprise a drying agent/desiccant as an element placed in or on the insert plug. The IVA test unit may, without limitation, comprise a drying agent/desiccant as an element placed in or on the cover plate. In other words, a drying agent may be a compound of the housing. A drying agent may be a compound of the closure element. A drying agent may be a compound of the cover plate. A drying agent may be an element placed in at least one chamber and/or recess of the housing, a drying agent may be an element placed in at least one chamber and/or recess of the insert plug and/or a drying agent may be an element placed in at least one chamber and/or recess of the cover plate.

Therefore, at least one element of the IVA test unit, may have an integrated drying function in that a drying agent is part, a portion and/or a compound of the material of which the element is made of and/or in that the element provides a volume into which the drying agent may be positioned and/or filled. The drying agent may, without limitation, have the form of a powder, pearls, a sponge, a solid piece, a liquid or the like. It is an advantage to provide elements of the IVA test unit, which already provide a drying function so to absorb water vapor and/or water and to protect the test strip from degrading. Therefore, the number of elements of a test set can be reduced, as no extra package with a drying agent is required to be added into the pouch. In other words, it is not required to provide a separate package with a drying agent in a pouch in which the test set is packed. This may reduce the complexity of producing a test set, less assembly steps of the test set may be required, less waste is generated, less volume is required for each packed test set and more compact packed test sets can be provided, which increases the shipping efficiency due to the ability of stapling the test sets and less empty air-filled space is shipped. Further, the IVA test unit is very efficient as it intrinsically provides a high(er) number of functionalities.

The proper function of the test strip may in general be sensitive to moisture and therefore the addition of a drying agent and/or a water-vapor-impermeable packaging is advantageous or even required to guarantee the proper function. Drying agents may comprise silica gels and/or zeolites. Chemical bonding can be established using salts (e.g., CaCl2) and/or calcium oxide.

The IVA test unit may, without limitation, comprise at least one of the following features to support dry conditions in the IVA test unit: a water-vapor-impermeable material which is comprised as an integral material of the housing, the closure element and/or the cover plate; a water-vapor-impermeable upper layer sprayed and/or coated onto the housing, the closure element and/or the cover plate; a water-vapor-impermeable film, preferably wherein the a water-vapor-impermeable film encloses the entire IVA test unit.

In other words, a water-vapor-impermeable material may be comprised as an integral material of the housing, a water-vapor-impermeable material may be comprised as an integral material of the closure element and/or a water-vapor-impermeable material may be comprised as an integral material of the cover plate. A water-vapor-impermeable upper layer may be sprayed and/or coated onto the housing. A water-vapor-impermeable upper layer may be sprayed and/or coated onto the closure element and/or a water-vapor-impermeable upper layer may be sprayed and/or coated onto the cover plate. A water-vapor-impermeable film may enclose parts of the IVA test unit or the entire IVA test unit, i.e., 100% of the IVA test unit. A water-vapor-impermeable film may enclose parts of the test set or the entire set.

If a water-vapor-impermeable material is used to enclose and/or seal the IVA test unit and/or the test set, it may not be required to pack the IVA test unit and/or the test set into a pouch. The IVA test unit may be provided to a user with or without other parts of the test set. However, in most cases the IVA test unit is provided to a user with all required elements of the test set.

The housing may comprise a fixing mechanism configured to fix the position of the test strip in the insert tray, optionally wherein the fixing mechanism comprises at least one of: a hook as an element of the housing and a recess as an element of the test strip configured to snap into the hook; a spring element which is configured to fix the test strip by means of a spring force; an element of the housing configured to fix the test strip by means of a friction force.

The fixing mechanism may hence allow securing the test strip inside the housing when pushing the test strip into the insertion tray specifically without the requirement of an additional movement. Therefore, a method of producing an IVA test unit and/or a test set comprising the IVA test unit is very efficient as the number of production steps can be reduced. Moreover, the fixing mechanism may comprise an element that is formed as a single piece with the housing. Therefore, the housing may obtain a further function and/or it may not be required to add a further element for achieving this function, which makes the housing very efficient and at the same time very reliable since the test strip is not accidentally released from the housing.

One element of the fixing mechanism may be provided at one end of the insert tray, for example, at the end opposite to the insert hole. Alternatively, the element of the fixing mechanism is provided near the insert hole. The counter element of the fixing mechanism may be provided on one end of the test strip.

In all cases, the housing may, without limitations, comprise a recycled plastic and/or a compostable material. The housing may be a disposable/configured for single-use. Alternatively, the housing may be configured to be re-used. Therefore, the housing may be composed of a material that is resistant to an aggressive cleaning procedure using for example, acids, a caustic solution, ozone, and other aggressive agents. A very resistant material is, for example, PTFE, however being more expensive than other polymer materials such as acrylic materials, which may be used as a material and/or compound of the housing, for example. Such housings are therefore very eco-friendly.

According to another aspect, which may be considered the second aspect of the present disclosure, a method of producing an IVA test unit comprises the following steps: providing a single-piece housing for storing a test strip, wherein the housing comprises an insert tray and an insert hole for inserting the test strip into the insert tray; placing the test strip in the housing.

The method may involve all the corresponding features, technical effects and advantages, which are outlined together with the embodiments of the IVA test unit described herein. Specifically, the method is very efficient in view of machine capacity, time and financial investment. The method allows a fast production. The method further allows producing a very efficient product (IVA test unit or test set comprising the IVA test unit) that can be efficiently handled, operated and/or shipped. The method may be easily upscaled to increase the production volume with low efforts. The following optional features and/or embodiments involve all the corresponding features, technical effects and advantages, which are outlined together with the (above) described IVA test unit and/or test set and the corresponding embodiments thereof.

The providing of the housing may relate to a mere providing wherein the production process is not involved. Alternatively, the providing of the housing may comprise producing the housing as a single piece by injection molding and/or by additive manufacturing, specifically by 3D printing.

Injection molding and/or additive manufacturing, specifically 3D printing are production principles by which single-piece elements can be formed. While additive manufacturing is typically a layer-by-layer application technique, the injection molding requires a mold defining a negative of a form/shape into which a liquid material forming the housing can be filled that obtains the form/shape when cured. Addictive manufacturing has the potential to produce any shape, even very complex shapes. Injection molding is limited with respect to the shapes that can be formed. If holes, recesses and the like should be formed, the injection molding method requires negative elements (forms/shapes) which are positioned with respect to the mold. Then the liquid material is filled into the mold. A shape is generated which is defined by the negative shape of the molding and the negative elements placed therein. Both principles are simple, can be easily automated and can efficiently allow producing single-piece elements at low costs while the upscaling of the production volume is also simple and efficient to realize.

When applying the technology of injection molding, it is an advantage to produce a single-piece housing, as only one single mold is required instead of several molds for each piece. At the same time, injection molding may require a complex-shaped form acting as a mold depending on the complexity of the housing shape. The mold may comprise a metal form that can be assembled before a molten, fluid and hot polymer is filled in and disassembled after the polymer has cooled down. The complex form may be shaped by a mold structure “from the outside” of the housing shape and “from the inside.” In other words, the mold may comprise a shaped cavity, defining the outside surface of the housing and elements which reach into the cavity and which define an inside surface of the housing, such as the insert tray and possibly the fixing mechanism. The size of such a mold and its structures is in the cm-range, i.e., between approximately 0.3 cm and approximately 30 cm, preferably between approximately 1 cm and 15 cm.

The placing of the test strip in the housing may comprise a pushing movement of the test strip into the housing. Further, the placing of the test strip in the housing may involve the following steps: providing a band and/or a sheet (of multiple connected test strips) with a longitudinal band/sheet axis, preferably from a roll, wherein the band/sheet comprises the test strip among a plurality of test strips along the longitudinal band axis; transporting the band, for example, on a conveyor, along the longitudinal band axis; cutting perpendicular to the longitudinal band axis the band/sheet to separate the test strip from the plurality of test strips; aligning the housing with a longitudinal housing axis parallel to or perpendicular to the longitudinal band axis and with the insert hole facing the test strip; and inserting the test strip into the insert tray through the insert hole.

The housing may, without limitation, be provided and/or moved by means of a conveyor, e.g., a conveyor belt. The method is very efficient and no pick-and-place is required. It is specifically not required to use a robot, which performs complex movements such as turning and/or twisting movements.

This embodiment of the method may be at least partially automated, specifically fully automated. The step of inserting the test strip into the insert tray may comprise, without limitation, a movement specifically a pushing movement by means of a piston, a hook, an airflow, a conveyor and/or the like.

The method may comprise closing the insert hole, without limitation, by at least one of the following: plugging an insert plug into the insert hole; clipping a cap to the housing over the insert hole.

The method may comprise providing a drying agent, without limitation, as at least one of the following: a compound of the housing, the insert plug and/or the cap; an element placed in at least one chamber and/or recess of the housing, the insert plug and/or the cap.

The method may comprise, without limitation, at least one of the following: spraying and/or coating a water-vapor-impermeable upper layer onto the housing; enclosing the entire IVA test unit with a water-vapor-impermeable film.

Closing the insert hole and/or providing a drying agent as a compound of the housing, another element and/or a coating/film may allow preserving the proper function and reliability of the test strip which may be sensitive to water and/or water vapor. Further, the number of elements which are required to ship and/or store the IVA test unit may be reduced and therefore, a very compact and/or efficient IVA test unit may be provided.

The method may comprise the step of fixing the position of the test strip in the insert tray using a fixing mechanism based, without limitation, on a snapping mechanism, a spring force and/or a friction force.

This method may allow reducing the number of production steps as the fixing may take place upon insertion and a further fixing step may not be required. Further, an IVA test unit may be produced in which the test strip is secured from moving, displacement and/or falling out of the housing, which makes the IVA test unit very reliable.

According to another aspect, which may be considered the third aspect of the present disclosure, a test set may comprise: the IVA test unit and/or any one of the embodiments of the IVA test unit described herein; a swab for collecting a biological sample; and a vial comprising a liquid for eluting the sample.

The test set (also denoted test kit) may further comprise a drying agent which is packed in a pouch and/or a water-vapor-impermeable pouch for packing the elements of the test set and/or a manual for providing guidance to a user as how to use the test set.

The housing may comprise at least one holder and/or at least one recess for holding and/or receiving one, several or all elements of the test set. The housing may comprise a vial holder and/or a vial recess to receive and/or hold the vial. Alternatively or in addition, the housing may comprise a swab holder and/or swab recess to receive and/or hold the swab. The vial holder and/or vial recess may be formed integrally with the housing, i.e., as a single piece with the housing. The swab holder and/or swab recess may be formed integrally with the housing, i.e., as a single piece with the housing. This makes it easy to produce a housing with multiple functions, assemble and/or keep together the essential elements of a test set. Further, it may improve the user's experience with the test set as the vial may, for example, be placed upright in a vial recess and therefore the user is not required to permanently hold the vial once it is opened. Moreover, the user finds all elements in a predetermined position at the housing which makes it easier to handle and which increases the user satisfaction. In a very compact version, a vial recess and a swab recess are provided in the housing such that the vial and the swab behave like inlays and are therefore flush with the general surface of the housing.

In general, the test set described herein corresponds to the set of elements, which may be provided to a user who wishes to perform a certain test to determine whether or not an analyte is present in a biological sample. For example, a test set may, without limitation, comprise an IVA test unit (a housing with a test strip and potentially other elements associated with the housing and/or the test strip such as a plug for closing an opening of the housing), a swab for collecting the sample, a liquid such as a buffer solution in a vial with application feature, such as a nozzle provided in the cap of the vial, a drying agent and a pouch into which all elements are packed, wherein the pouch may be considered as being a part of the set. The pouch has the function of maintaining dry conditions inside the pouch and preventing the test strip from degradation. In some embodiments, the housing or an element associated and/or being connectable with the housing may comprise a drying agent. In that case, for example, the test set does not require an additional drying agent, which is provided as a separate element.

The IVA test unit described herein comprises at least the single-piece housing and the test strip which may be placed inside the housing. The test strip may also be delivered to a user or a distributor without being placed inside the housing. The housing and the strip may be provided as separate elements however, most likely, the IVA test unit is already assembled when provided to the user, i.e., the test strip is positioned inside the housing.

In the following, some example embodiments will be described in detail, wherein this disclosure should not be understood to be limited to the example embodiments described. The following examples and figures are provided to aid the understanding of this disclosure, the true scope of which is set forth in the appended claims. Single features being described in a particular embodiment may be arbitrarily combined, given that they are not excluding each other. In addition, different features which are provided together in the example embodiments are not to be considered restrictive to this disclosure. Skilled artisans appreciate that elements in the figures are illustrated for simplicity and clarity and have not necessarily been drawn to scale. For example, the dimensions of some of the elements in the figures may be exaggerated relative to other elements whereas other elements may have been left out or represented in a reduced number in order to enhance clarity and improve understanding of the aspects of the present disclosure. The same reference numerals are used among different embodiments and examples for the same or similar elements or elements that have similar or the same effects.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 shows several elements of a lateral flow assay test set/kit according to the prior art;

FIG. 2 is a schematic drawing showing, in a perspective side view, the assembly steps of an IVA test unit comprising a housing and a test strip according to one embodiment;

FIG. 3 is a schematic drawing showing the method steps of producing an IVA test unit according to one embodiment;

FIG. 4 is a schematic drawing showing the method steps of placing the test strip into the housing according to one embodiment;

FIG. 5 shows an IVA test unit comprising a transparent housing with an application recess according to one embodiment;

FIG. 6 shows an IVA test unit comprising a transparent housing with a cap, an application recess and a recess in the control area according to one embodiment;

FIG. 7 shows an IVA test unit comprising a housing with an application recess and a recess in the control area packed in a water-vapor-impermeable foil according to one embodiment;

FIG. 8 shows an IVA test unit comprising a housing with an application recess and a recess in the control area wherein the housing is coated with a water-vapor-impermeable material according to one embodiment;

FIG. 9a is a schematic drawing showing a top view of an IVA test unit comprising a test strip and a housing according to one embodiment;

FIG. 9b is a schematic drawing showing a front view of the IVA test unit of FIG. 9a along the line AA′ according to one embodiment;

FIG. 9c is a schematic drawing showing a top view of a test set with a vial, a swab, and an IVA test unit comprising a test strip and a housing according to one embodiment;

FIG. 9d is a schematic drawing showing a cut of the test set of FIG. 9c along the cut line BB′ according to one embodiment;

FIG. 9e is a schematic drawing showing a top view of a test set with a vial, a swab, and an IVA test unit comprising a test strip and a housing according to one embodiment;

FIG. 9f is a schematic drawing showing a cut of the test set of FIG. 9e along the cut line CC′ according to one embodiment;

FIG. 9g is a schematic drawing showing a top view of a test set with a vial, a swab, and an IVA test unit comprising a test strip and a housing according to one embodiment;

FIG. 9h is a schematic drawing showing a cut of the test set of FIG. 9g along the cut line DD′ according to one embodiment;

FIG. 10a is a schematic drawing showing a top view of a housing with a cap according to one embodiment;

FIG. 10b is a schematic drawing showing a front view of the housing of FIG. 10a along the line EE′ according to one embodiment;

FIG. 11a is a schematic drawing of the IVA test unit in a perspective side view having an insert tray positioned lower than the insert hole, while the insert hole is tapered from the outer side towards the inner side to reduce the risk of losing the test strip via the insert hole according to one embodiment;

FIG. 11b is the schematic drawing of the IVA test unit of FIG. 11a in a front view;

FIG. 12a is a schematic drawing of the IVA test unit in a perspective side view having an insert hole tilted with respect to a plane parallel to the upper and lower walls of the housing to reduce the risk of losing the test strip via the insert hole according to one embodiment;

FIG. 12b is a schematic drawing of the IVA test unit of FIG. 12a in a front view;

FIG. 13 is a schematic drawing of the IVA test unit in a side view having a flap in front of the insert hole to reduce the risk of losing the test strip via the insert hole according to one embodiment;

FIG. 14 is a schematic drawing of the IVA test unit in a front view having a curved inner side of the insert hole, while the insert hole is tapered from the outer side towards the inner side, to reduce the risk of losing the test strip via the insert hole according to one embodiment;

FIG. 15a is a schematic drawing of the IVA test unit in a perspective side view having an insert tray positioned lower than the insert hole and the lower surface of the insert tray is tilted with respect to the upper wall of the housing according to one embodiment;

FIG. 15b is a schematic drawing of the IVA test unit in a perspective side view having an insert tray positioned lower than the insert hole and on the lower surface of the insert tray a support structure is provided according to one embodiment; and

FIG. 15c is a schematic drawing of the IVA test unit in a perspective side view having an insert tray positioned lower than the insert hole and the upper surface of the insert tray is tilted with respect to the lower wall of the housing according to one embodiment.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

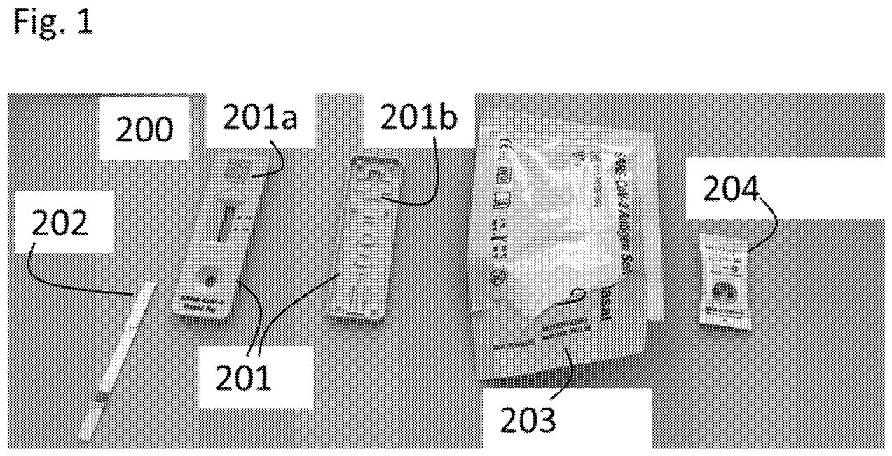

FIG. 1 shows several elements of a lateral flow assay test (LFA) set/kit 200 according to the prior art (denoted prior art LFA-set 200). Specifically, FIG. 1 shows a rapid antigen test for detecting the presence of Covid 19 in a biological sample. The prior art LFA-set 200 comprises a test strip 202, which is an LFA-core strip 202, a housing 201 which comprises two pieces, namely an upper 201a and a lower piece 201b, a package with a drying agent 204, a swab (not shown) and a water-vapor-impermeable pouch/packaging 203. As the housing comprises two pieces 201a, 201b, there is a risk that the housing disassembles by mistake, for example, when the coupling mechanism, that couples the upper 201a with the lower piece 201b, is damaged and/or when an external force impacts the housing 201. Typically, the prior art LFA-set 200 is assembled using a pick-and-place method, which requires multiple steps and which may be time-consuming and/or complex in terms of automated movements. As the prior art LFA-set 200 comprises multiple elements, it may be complex, time-consuming and/or difficult to assemble and pack the set. Further, the packed prior art LFA-set 200 is not very compact and adopts an undefined shape such that stacking is not easy or not possible. In addition, a lot of empty air-filled space is packed. This disclosure was made to generally improve IVA test units and/or test sets, specifically in view of the above-described difficulties.

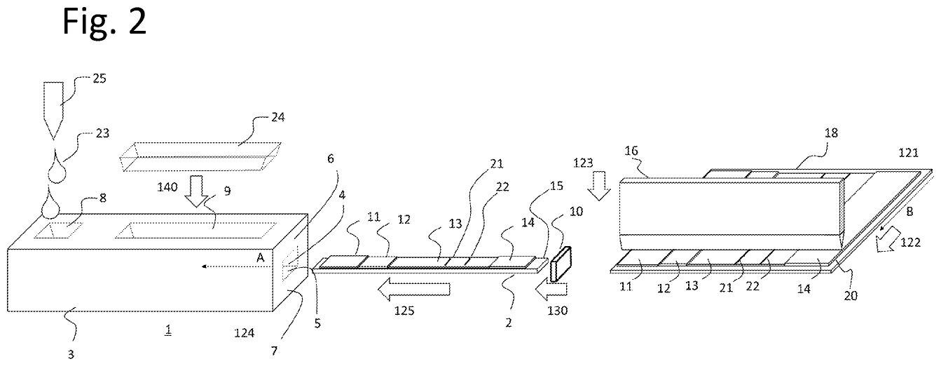

FIG. 2 is a schematic drawing showing the assembly steps of an IVA test unit 1 comprising a housing 3 and a test strip 2, according to one embodiment. The housing 3 is a single-piece housing for storing a test strip 2. The housing 3 comprises an insert tray 4 and an insert hole 5 for inserting the test strip 2 into the insert tray 4. In this embodiment, the insert hole 5 corresponds to the opening of the insert tray 4 and the cross section area of the insert hole 5 corresponds to the cross section area of the insert tray 4. Alternatively, the insert opening 5 may have a smaller cross section area than the insert tray 4 and may, for example, be positioned slightly higher than the lower surface of the insert tray 4.

The insert hole 5 is positioned on a short side of the housing 3 at a surface that is substantially perpendicular to a longitudinal axis A of the housing 3. The housing 3 further comprises a first wall 6, which is illustrated in FIG. 2 as an upper wall and a second wall 7 which is illustrated in FIG. 2 as a lower wall. Overall, in this embodiment, six side surfaces and/or side walls, which lie in planes that are perpendicular to each other, are provided. The first and the second wall 6, 7 face each other, i.e., the walls 6, 7 are adjacent each other and define the insert tray 4 at least partially by enclosing the insert tray 4 on the upper and the lower sides. The first wall 6 comprises an application recess 8 and the second wall 7 is configured to support the test strip 2 from underneath when positioned in the correct upright orientation. The housing 3 may specifically be produced as a single piece by injection molding or by additive manufacturing, specifically by 3D printing. The housing 3 comprises the application recess 8 for allowing the application of a sample liquid 23 which may be applied by a tip 25 of a needle or a pipette onto the test strip 2 when the test strip 2 is positioned inside the insert tray 4. The sample liquid 23 may comprise the biological sample collected with a swab eluted in the liquid. The liquid may be an aqueous buffer solution provided in a vial. Moreover, the housing 3 comprises a recessed control area 9 for reading out a test result from the test strip 2 when the test strip 2 is positioned inside the insert tray 4, the sample liquid 23 is applied and the test is ready for being read out.

According to FIG. 2, a band and/or sheet 18 with a longitudinal band/sheet axis B is provided 121 and transported 122 on a conveyor along the longitudinal band axis B. The band 18 comprises the test strip 2 among a plurality of test strips 20 along the longitudinal band axis B. The longitudinal band axis B is perpendicular to the longitudinal axis of a single test strip 2 among the plurality of test strips 20. In further detail, the test strips 20 are provided in the form of a band/sheet. The test strips 20 in the form of a band/sheet 18 already comprise all elements, such as a substrate 15, a sample pad 11, a conjugate pad 12, a membrane 13, such as a nitrocellulose membrane and an absorption pad 14. The plurality of test strips 20 of the sheet are cut 123 by a cutter 16 in a perpendicular direction to the longitudinal band axis B to obtain a single test strip 2. This cutting direction is used if the test strips 20 are connected to each other side by side.

Alternatively, the test strips 20 may be provided one by one being neighbored on the test strip's 20 short side (not shown in the figure). In that case, the longitudinal band axis B corresponds to the longitudinal axis of a single test strip 2 among the plurality of test strips 20.

In the present FIG. 2, the housing 3 is aligned 124 with the longitudinal housing axis A perpendicular to the longitudinal band axis B and with the insert hole 5 facing the test strip 2. The longitudinal axis of a single test strip 2 is aligned along the longitudinal housing axis A. The test strip 2, after it was cut off the band of test strips 20, is inserted 125 into the insert tray 4 of the housing 3 through the insert hole 5.

An insert plug 10 is plugged 130 into the insert hole 5, after the insertion 125. Alternatively, a cap 30 may be clipped over the insert hole 5 onto the housing 3. Moreover, the IVA test unit 1 is sealed 140 by means of a cover plate 24 that is inserted into the recessed control area 9.

The final product produced by the method shown in FIG. 2 should be an IVA test unit 1 that has a test strip 2 positioned inside the insert tray 4 wherein the sample pad 11 is substantially aligned with the application recess 8 and a test line 21 and a control line 22 of the membrane 13 are aligned with the control area 9. In other words, the sample pad 11 should be under the application recess 8 such that a user can reach the sample pad 11 via the application recess 8 to apply a sample liquid 23. Further, the test line 21 and the control line 22 of the membrane 13 should be visible through the control area 9.

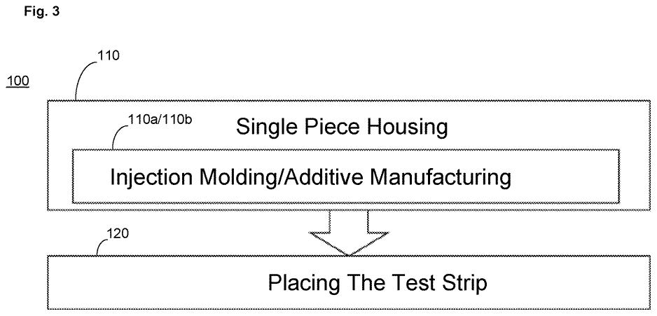

FIG. 3 is a schematic drawing showing the method steps of producing 100 the IVA test unit 1 according to one embodiment. The method 100 of producing the IVA test unit 1 comprises the following steps: providing 110 the single-piece housing 3 for storing the test strip 2, wherein the housing 3 comprises an insert tray 4 and an insert hole 5 for inserting the test strip 2 into the insert tray 4 and placing 120 the test strip 2 into the insert tray 4 of the housing 3. The providing of the housing 3 comprises in this embodiment a producing of the housing 3 as a single piece by injection molding 110a and/or by additive manufacturing 110b, specifically by 3D printing. The providing of the housing 3 may in addition and/or as an alternative refer to the mere providing of the housing 3 to a user, a supplier and/or a manufacturer. The housing 3 may be provided as part of an IVA test unit 1 or a test set 17.

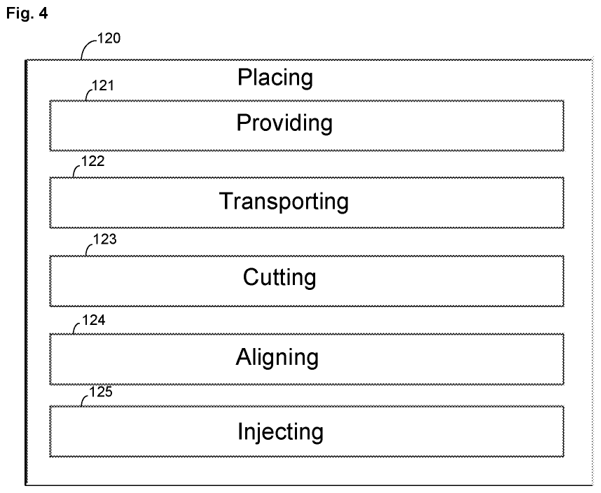

FIG. 4 is a schematic drawing showing the method steps of placing 120 the test strip 2 in the housing 3 according to one embodiment. The placing of the test strip 2 into the housing 3 comprises providing 121 a band/sheet 18 with a longitudinal band axis B, preferably from a roll, wherein the band/sheet 18 comprises the test strip 2 among a plurality of test strips 20 along the longitudinal band axis B; transporting 122 the band/sheet 18 on a conveyor along the longitudinal band axis B; cutting 123 perpendicular to the longitudinal band axis B the band/sheet 18 to separate the test strip 2 from the plurality of test strips 20; aligning 124 the housing 3 with a longitudinal housing axis A in line (when the band axis B is in line with the housing axis A), parallel to or perpendicular to (when the band axis B is perpendicular to the housing axis A as shown in FIG. 2) the longitudinal band axis B and with the insert hole 5 facing the test strip 2; and inserting 125 the test strip 2 into the insert tray 4 through the insert hole 5. In all cases, the housing axis A should substantially align with the longitudinal axis of a single test strip 2 when the test strip 2 is inserted into the insert tray 4.

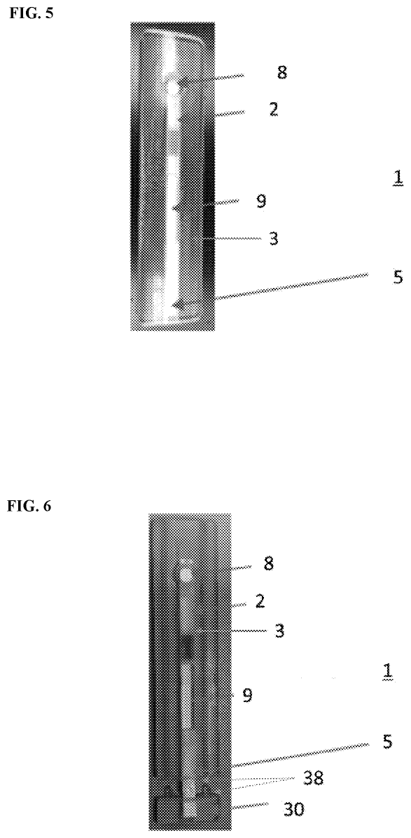

FIG. 5 shows an IVA test unit 1 comprising a transparent housing 3 with an application recess 8 and a recess in the control area 9, according to one embodiment. In this specific embodiment, the transparent housing 3 comprises a recess in the control area 9. However, as the housing 3 is transparent, a recess in the control area 9 is not required for a user to be able to read the test result. The test strip 2, which is an LFA-core strip, would also be visible through the transparent housing 3 if the recess in the control area 9 would not be provided, which makes it easy for a user to read the test result from the test strip 2. The sample liquid may be applied onto the sample pad 11 of the test strip 2 through the application recess 8.

The proper function of the test strip 2 may in general be sensitive to moisture/water vapor and/or water and therefore requires the addition of a drying agent. In this specific embodiment of FIG. 5, the housing 3 does not comprise a drying agent as a compound of the housing 3. It may be required to add a pouch with a drying agent to the IVA test unit 1 to maintain dry conditions. Alternatively to this embodiment, the housing 3 may comprise a drying agent. It may therefore not be required to add a package/pouch with a drying agent to a bigger package/pouch together with the IVA test unit 1. Alternatively or in addition to providing a pouch, the application recess 8 and/or the recess in the control area 9 may be covered, particularly sealed by a cover plate and/or a foil. At least one foil/film may be glued, welded and/or laminated to the housing covering one or both of the recesses. In such a case, the cover plate and/or the foil/film may comprise a drying agent to absorb the moisture in the internal volume, specifically the insert tray 4 of the housing 3 and to protect the function of the test strip 2.

FIG. 6 shows an IVA test unit 1 comprising a transparent housing 3 with a cap 30 that comprises a drying agent, with an application recess 8 and a recess in the control area 9 according to one embodiment. The housing 3 may have the same features as the housing 3 of the embodiment shown in FIG. 5. In addition, the IVA test unit 1 of FIG. 6 comprises a coupling mechanism which allows to couple, fix and/or attach the cap 30 to the housing 3. The housing 3 of FIG. 6 therefore comprises one component of the coupling mechanism 38 and the cap 30 comprises the counter-element of the coupling mechanism 38. More specifically, the cap 30 comprises two extending elements (noses), which face the housing 3 when the cap 30 is about to be coupled to the housing 3. The extending elements fit two corresponding recesses (not visible in this figure) in the housing 3. The extending elements on the cap 30 and the corresponding recesses in the housing form the coupling mechanism 38. The cap 30 may not be considered as being part of the housing 3. The cap 30 has a slit, which aligns with the insert tray 4 of the housing for receiving one end of the test strip 2. The cap 30 covers and thereby closes the insert hole 5 of the housing 3. The cap 30 comprises an integral drying agent. Alternatively or in addition, the cap 30 may have an internal volume that may house the drying agent, wherein the internal volume can be connected to the insert tray 4 when the cap 30 is attached to the housing 3. In general, the application recess 8 and/or the recess in the control area 9 may be covered, particularly sealed by a cover plate and/or a foil.

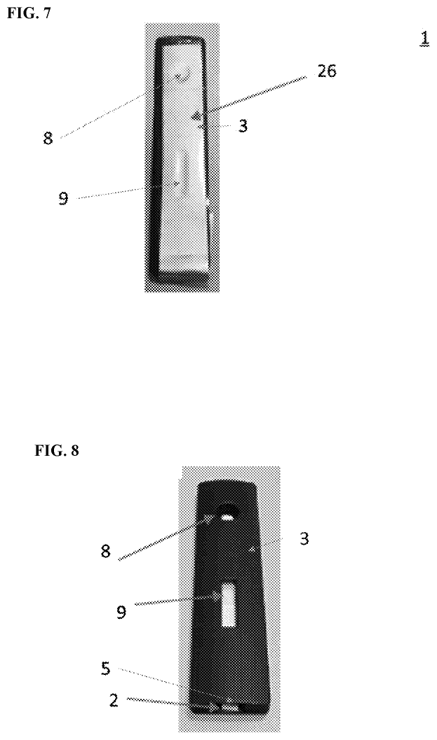

FIG. 7 shows an IVA test unit 1 comprising a housing 3 with an application recess 8 and a recess in the control area 9 packed in a transparent water-vapor-impermeable foil 26 according to one embodiment. The transparent water-vapor-impermeable foil 26 allows a user to see and/or check the state of the IVA test unit 1. At the same time, the transparent water-vapor-impermeable foil 26 protects the IVA test unit 1 from moisture, water and/or water vapor. In this embodiment, the IVA test unit 1 is tightly packed in the transparent water-vapor-impermeable foil 26.

FIG. 8 shows an IVA test unit 1 comprising a housing 3 with an application recess 8 and a recess in the control area 9 wherein the housing 3 is coated with a water-vapor-impermeable material according to one embodiment.

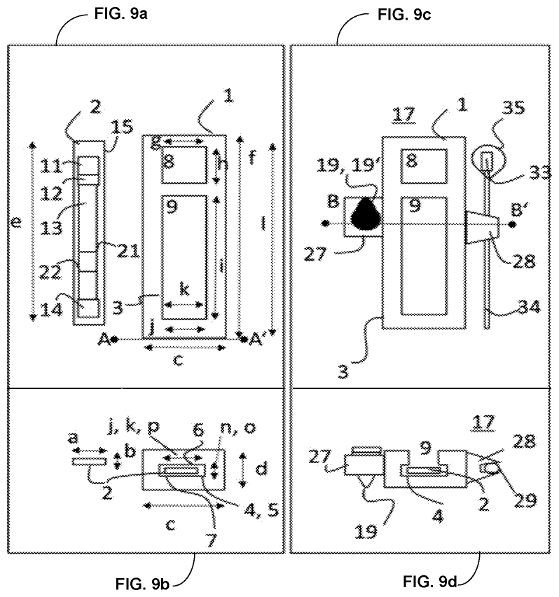

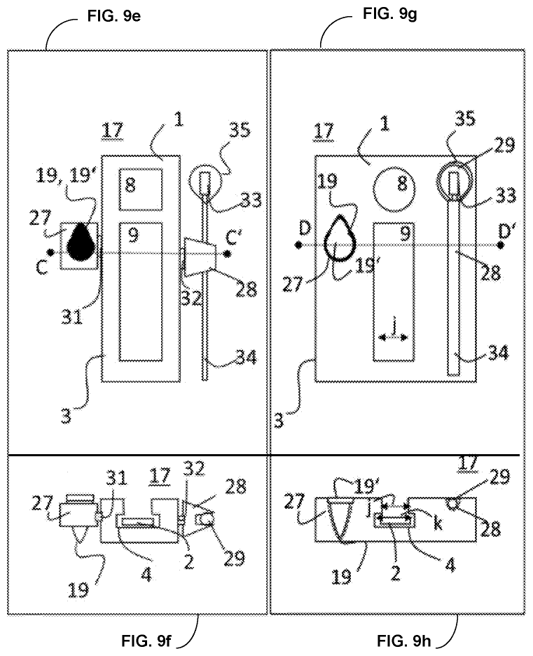

In the embodiments shown in FIG. 5, FIG. 8 and FIG. 9, all recesses and the insert hole 5 are open; therefore, it may be advantageous to provide a cap 30 or an insert plug 10 (possibly together with a coupling mechanism to couple the insert plug to the housing 3) for closing and/or sealing the insert hole 5 and it may be advantageous to provide a cover plate and/or a foil to cover and/or seal the application recess 8 and/or the recess in the control area 9. At least one foil/film may be glued, welded and/or laminated to the housing covering one or both of the recesses 8, 9. The cover plate and/or foil may be water-vapor-impermeable and/or transparent. In general, the cover plate, foil and/or film may comprise a drying agent and/or a chamber/internal volume for housing the drying agent.

FIG. 9a is a schematic drawing showing a top view of an IVA test unit 1 comprising a test strip 2 and a housing 3 according to one embodiment. FIG. 9b is a schematic drawing showing a front view of the IVA test unit 1 of FIG. 9a along the line AA′ that indicates the front of the housing 3.

In both figures, the test strip 2 (which is an LFA-core strip) is shown separately on the left side. On the right side, the housing 3 is shown in which an LFA-core strip 2 is inserted in the insert tray 4. In this embodiment, the insert hole 5 and the insert tray 4 have the same cross section area and align with each other-in other words, the insert hole 5 is the opening of the insert tray 4.

The test strip 2 is a typical LFA-core strip and comprises-as mentioned previously-a substrate 15 on which several elements are positioned. A sample pad 11 is positioned on one side of the substrate 15 to receive the sample liquid that is typically applied through the application recess 8 of the housing 3 when the LFA-core strip 2 is correctly inserted in the housing 3. The sample pad 11 contacts a conjugate pad 12. Typically, the conjugation pad 12 contains detection reagents, i.e., conjugates. When the sample liquid flows through the conjugate pad, the conjugate is released into the sample liquid and binds with the target molecule, if present in the sample liquid. The conjugation pad 12 contacts a membrane 13 such as a nitrocellulose membrane. When the sample liquid/fluid passes the membrane 13, the result is visualized on the membrane 13 by means of a control line 22 and—if the target molecule is present—by means of a test line 21. In other words, if the target molecule is not present in the sample liquid, the test line 21 remains invisible or is not present. If the target molecule is present in the sample liquid, the test line 21 becomes visible or is present. In all cases, the control line 22 is visible once the sample liquid has passed the membrane.

The test strip 2 is positioned with the sample pad 11 substantially under the application recess 8 when being fully and correctly inserted into the insert tray 4. The application recess 8 is located in/at the housing 3 in a region away from the insert hole 5. For example, the insert hole 5 may be provided in the front of the housing 3 and the application recess 8 may be provided in the back of the housing 3. The control area 9 is located between the insert hole 5 and the application recess 8.

It may however also be the other way around and the insert hole 5 may be provided in the front of the housing 3 and the application recess 8 may also be provided in the front of the housing 3. Therefore, in an alternative embodiment, the application recess 8 may be located in/at the housing 3 in a region close to the insert hole and the control area 9 may then be located in/at the housing 3 in a region away/apart from the insert hole 5. In the latter case, the LFA-core strip needs to be inserted in the opposite direction as compared to the first case in which the application recess 8 is located in/at the housing 3 in a region apart from the insert hole 5.

In the following, the dimensions of some features of the housing 3 and the test strip 2 are specified. These dimensions should be understood as exemplary embodiments to better understand this disclosure and not as necessary limitations.

The test strip 2 has a length “e,” a width “a” and a height “b.” The length “e” may, without limitation, range between approximately 3 cm and 15 cm, specifically between approximately 4 cm and 10 cm and more specifically between approximately 5 cm and 8 cm. The width “a” may, without limitation, range between approximately 0.5 cm and 3 cm, specifically between approximately 0.8 cm and 2 cm and more specifically between approximately 0.9 cm and 1.5 cm. The height “b” may, without limitation, range between approximately 0.5 mm and 3 mm, specifically between approximately 0.8 mm and 2.5 mm and more specifically between approximately 0.9 mm and 2 mm.

The housing 3 is shown in FIG. 9a and FIG. 9b on the right side, wherein a test strip 2 is placed in the insert tray 4, as can be seen in FIG. 9b. In FIG. 9a, the housing 3 is schematically shown from a top view. The figure shows details such as the application recess 8, the control area 9 and the structure of the housing 3. The control area 9 may, without limitation, be realized as a recess in the housing 3. Alternatively, the control area 9 may, without limitation, be realized as a transparent portion of the housing 3, a window, a foil and/or a film. In addition or alternatively, the application recess 8 and/or the control area 9 may, without limitation, be covered and/or closed by a cover plate, a film and/or a foil, specifically to seal the inside volume of the housing 3. Further, the insert hole 5 may, without limitation, be covered, sealed and/or closed by an insert plug 10, a cap 30, a film and/or a foil.

The housing 3 has a length “f,” a width “c” and a height “d.” The length “f” may, without limitation, range between approximately 4 cm and 20 cm, specifically between approximately 5 cm and 15 cm and more specifically between approximately 6 cm and 10 cm.

The insert tray 4 has a length “l,” a width “k” and a height “n.” The length “f” may, without limitation, range between approximately 3.9 cm and 19.9 cm, specifically between approximately 4.9 cm and 14.9 cm and more specifically between approximately 5.9 cm and 9.9 cm.

The housing 3 may, without limitation, have a length “f” that is longer than “e.” In some other cases, specifically when a cap 30 is provided that also houses a portion of the test strip 2, the length “f” of the housing 3 may be identical with or shorter than the length “e” of the test strip 2. In such cases, the length “f” of the housing 3 may, without limitation, be shorter than the length “e” of the test strip 2 by approximately 0 cm to 5 cm, specifically by approximately 0.4 cm to 2 cm and more specifically by approximately 0.5 cm to 1.5 cm. Alternatively, the length “f” of the housing may, without limitation, be longer than the length “e” of the test strip 2 by approximately 0.3 cm to 5 cm, specifically by approximately 1 cm to 2 cm and more specifically by approximately 1.2 cm to 1.5 cm.

The insert tray 4 may, without limitation, have a length “l” that is longer than the length of the test strip “e.” In some other cases, specifically when a cap 30 is provided that also houses a portion of the test strip 2, the length “l” of the insert tray 4 may be identical with or shorter than the length “e” of the test strip 2. In such cases, the length “l” of the insert tray 4 may, without limitation, be shorter than the length “e” of the test strip 2 by approximately 0 cm to 5 cm, specifically by approximately 0.4 cm to 2 cm and more specifically by approximately 0.5 cm to 1.5 cm. Alternatively, the length “l” of the insert tray 4 may, without limitation, be longer than the length “e” of the test strip 2 by approximately 0.3 cm to 5 cm, specifically by approximately 1 cm to 2 cm and more specifically by approximately 1.2 cm to 1.5 cm.

The width “c” of the housing 3 may, without limitation, range between approximately 0.8 cm and 5 cm, specifically between approximately 0.9 cm and 3 cm and more specifically between approximately 1 cm and 2.5 cm. The height “d” of the housing 3 may, without limitation, range between approximately 0.8 cm and 3 cm, specifically between approximately 1 cm and 2.5 cm and more specifically between approximately 1.3 cm and 2 cm.

The width “k” of the insert tray 4 may, without limitation, range between approximately 0.51 cm and 3.1 cm, specifically between approximately 0.81 cm and 2.1 cm and more specifically between approximately 0.91 cm and 1.51 cm. The height “n” of the insert tray 4 may, without limitation, range between approximately 0.7 cm and 2.9 cm, specifically between approximately 0.9 cm and 2.4 cm and more specifically between approximately 1.2 cm and 1.9 cm. In other words, the insert tray 4 may, without limitation, have a length “l,” a width “k” and a height “n,” which depend on the thickness of the walls of the housing 3. The dimensions of the insert tray 4 may therefore, without limitation, correspond to the according dimensions of the housing 3 minus the thickness of the housing walls at the certain position. The thickness of the housing walls may, without limitation, range between approximately 0.05 cm and 2 cm, specifically between approximately 0.2 cm and 1 cm and more specifically between approximately 0.25 cm and 0.4 cm.

The application recess 8 may have, without limitation, an arbitrary shape from a top view. In the present embodiment, the shape of the application recess 8 is rectangular, specifically squared. Alternatively, the application recess 8 may, without limitation, be circular, elliptic or polygonal. The sidewalls of the housing 3 extending from the application recess 8 downwards to the insert tray may, without limitation, be tapered or may extend perpendicularly from the upper surface of the housing 3. The application recess 8 has a length “h,” a width “g” and a depth (not indicated). The depth typically corresponds to the thickness of the housing wall. The length “h” may range between approximately 0.3 cm and 4 cm, specifically between approximately 0.5 cm and 3 cm and more specifically between approximately 0.8 cm and 2 cm. The width “g” may range between approximately 0.3 cm and 3.5 cm, specifically between approximately 0.5 cm and 2.8 cm and more specifically between approximately 0.8 cm and 1.5 cm.

The control area 9, which may correspond to a recess in the housing 3 may have an arbitrary shape from a top view. In the present embodiment, the shape of the control area 9 is rectangular. Alternatively, the control area 9 may, without limitation, be elliptic or polygonal. In the case of a recess present in the control area 9, the sidewalls of the housing 3 extending from the control area 9 downwards to the insert tray may, without limitation, be tapered or may extend perpendicularly from the upper surface of the housing 3.

The control area 9 has a length “i,” a width “j” and possibly a depth if a recess is provided in the housing 3. The depth typically corresponds to the thickness of the wall of the housing 3 in that position. The length “i” may, without limitation, range between approximately 1 cm and 10 cm, specifically between approximately 2 cm and 8 cm and more specifically between approximately 3 and 6 cm. The width “j” may, without limitation, range between approximately 0.3 cm and 3.5 cm, specifically between approximately 0.5 cm and 2.8 cm and more specifically between approximately 0.8 cm and 1.5 cm. The width “j” of the control area 9 may, without limitation, be identical with the width “g” of the application recess 8. Further, the width “j” of the control area 9 and the width “g” of the application recess 8 may be identical with or smaller than the width of the insert tray 4.

The width “p” and/or the height “o” (also denoted “slit size”) of the insert hole 5 may, without limitation, respectively correspond to the width “k” and/or the height “n” of the insert tray 4. Alternatively, for example, when the insert hole 5 is tapered towards the insert tray 4, the width “p” and/or the height “o” of the insert hole 5 may respectively be larger or smaller than the width and/or the height of the insert tray 4. Further, without limitation, the width “p” of the insert hole 5 and/or the width “k” of the insert tray 4 may correspond to the width “j” of the control area 9 and/or the width “g” of the application recess 8 (see, for example, FIGS. 9a and 9b). Alternatively, the width “p” of the insert hole 5 and/or the width “k” of the insert tray 4 may be smaller than the width “j” of the control area 9 and/or the width “g” of the application recess 8 (see, for example, FIG. 9c to 9h).

As illustrated in some embodiments of the figures, the application recess 8 and the control area 9 correspond to two isolated recesses in the housing 3. Alternatively, the application recess 8 and the control area 9 may correspond to one single recess in the housing 3 (not shown in any embodiment).

In FIG. 9b, the first wall 6 and the second wall 7 are shown. The first wall 6 corresponds to the upper wall of the housing 3 and the second wall 7 corresponds to the lower wall of the housing 3. The first wall 6 and the second wall 7 are facing each other and enclose at least partially the insert tray 4. In the presently shown cross section along AA′, the first and the second wall 6, 7 enclose the insert tray 4 together with the side walls. Slightly further above, in the region of the recess of the control area 9 or in the region of the application recess 8, the upper wall is not closed and recessed, as can be seen in the following FIGS. 9d, 9f and 9h where the recess of the control area 9 is visible. The recesses are not indicated to be tapered in the shown embodiments but may be tapered in other embodiments.

The specifications mentioned for the embodiment shown in FIG. 9a and FIG. 9b may apply to other embodiments described herein.

FIG. 9c is a schematic drawing showing a top view of a test set/test kit 17, namely an IVA test unit 1 comprising a test strip 2, a housing 3, a vial holder 27, a vial 19, a swab holder 28 and a swab 29 having a stick piece 34 and a head piece 33 which is wrapped and/or packed in a packaging 35, according to one embodiment. FIG. 9d is a schematic drawing showing a cut of the test set/test kit 1 of FIG. 9c along the cut line BB′. The center portion of the housing 3 shown in this embodiment may have the dimensions of to the housing 3 shown in FIG. 9a and FIG. 9b or may have similar dimensions as the housing 3 shown in FIG. 9a and FIG. 9b.

The vial holder 27 and/or the swab holder 28 may be formed as a single piece with the center portion of the housing 3. Alternatively, the vial holder 27 and/or the swab holder 28 may be formed as a separate piece/separate pieces being coupled to the housing 3.

The vial holder 27 of this embodiment has a squared outer shape and a round recess to receive the vial 19 that has a round shape in its cross section. The outer contour/shape of the vial holder 27 has a square shape. In general, the outer contour/shape of the vial holder 27 and/or the recess shape may differ from the shown shapes. Both shapes may comprise polygonal, elliptic, round, squared or other shapes.

The swab holder 28 of this embodiment has the shape of a trapezoid from the top view with a recess that is configured to receive and hold the swab 29. The swab holder 28 may alternatively have a different shape, such as a polygonal, a round, an elliptic and/or a square shape. As it becomes visible in the cut view (FIG. 9d), the swab holder 28 of this embodiment extends in a tapered manner from the housing 3. The recess configured to receive and hold the swab 29 may correspond the shape of the stick piece 34 of the swab 29. For example, the cross section of the stick piece 34 of the swab 29 may be round or polygonal and the recess configured to receive and hold the swab 29 may therefore correspond to this round or polygonal shape of the stick piece 34. The swab holder 28 may be flexible and/or resilient such that a force presses against the stick piece 34 to hold and/or fixate the swab 29 to a certain extent. The swab 29 can however be released from the swab holder 28 as the force applied by the swab holder 28 onto the stick piece 34 is not too strong. Therefore, when shipping the test set/test kit 17, the swab 29 and the vial 19 may be coupled to the according holders 27, 28. In the moment when the test set/test kit 17 is used, the swab 29 and/or the vial 19 may be removed from their holders 27, 28. Specifically, the swab 29 may be removed to collect the biological sample. The vial 19 may however remain in the vial holder 27 and merely the vial cap 19′ may be removed to open the vial 19 such that the (flexible porous) head piece 33 of the swab 29 can be dipped into the liquid which is stored in the vial 27.

Similarly as in FIG. 9c, FIG. 9e is a schematic drawing showing a top view of a test set/test kit 17, namely an IVA test unit 1 comprising a test strip 2, a housing 3, a vial holder 27, a vial 19, a swab holder 28 and a swab 29 having a stick piece 34 and a head piece 33 which is wrapped and/or packed in a packaging 35, according to one embodiment. In this embodiment, the vial holder 27 and the swab holder 28 are respectively connected and/or coupled with the housing 3 by means of a bridge 31 for a vial holder 27 and a bridge 32 for a swab holder 28. Similarly as in FIG. 9d, FIG. 9f is a schematic drawing showing a cut of the test set/test kit 17 of FIG. 9e along the cut line CC′. The vial holder 27, the bridge 31 for the vial holder 27, the swab holder 28 and/or the bridge 32 for the swab holder 28 may be formed as a single piece with the housing 3. Alternatively, the bridge 31 for the vial holder 27, the swab holder 28 and/or the bridge 32 for the swab holder 28 may be formed as a separate piece/separate pieces being coupled to the housing 3. At least one of the bridges 31, 32 may be configured to be broken and/or detached by a user to release the vial holder 27 and/or the swab holder 28 from the housing 3. If one of the bridges 31, 32 can be broken this means that the according holder 27, 28 can be irreversibly detached from the housing 3. Therefore, the according bridge 31, 32 can have a breaking point that allows to easily break the bridge 31, 32. If one of the bridges 31, 32 can be reversibly detached from the housing 3, a coupling mechanism may be provided with one element provided at or in the housing 3 and the counter element provided at or in the bridge 31, 32. If no bridge 31, 32 is provided, as, for example, in the embodiment of FIG. 9c, the vial holder 27 and/or the swab holder 28 may also be detachable from the housing 3 and therefore, a coupling mechanism may be provided with one element provided at or in the housing 3 and the counter element provided at or in the according holder 27, 28.

At least one of the bridges 31, 32 may be flexible such that the vial holder 27 and/or the swab holder 28 can be twisted. If the bridge 31 for the vial holder 27 is detachable in a reversible or irreversible manner, the vial holder 27 may comprise a rim for placing the vial 19 with the vial holder 27 in an upright manner on a plane underground.

In the embodiments of FIG. 9c and FIG. 9e, the housing 3 consists of a block-like structure wherein the vial holder 27 and the swab holder 28 are either attached to the side of the housing 3 or formed integrally with the housing 3 but always extending from the block-like structure of the housing 3. In FIG. 9g, a different structure of the housing 3 is shown.

FIG. 9g is a schematic drawing showing a top view of a test set/test kit 17, namely an IVA test unit 1 comprising a test strip 2, a housing 3, a vial holder 27, a vial 19, a swab holder 28 and a swab 29 having a stick piece 34 and a head piece 33 which is wrapped and/or packed in a packaging 35, according to one embodiment. In this embodiment, the vial holder 27 corresponds in fact to a vial recess in the housing 3 and the swab holder 28 also corresponds to a swab recess in the housing 3. The housing 3 is substantially formed as a block with all recesses and/or holders being integrated in the housing 3. FIG. 9h is a schematic drawing showing a cut of the test set/test kit 17 of FIG. 9g along the cut line DD′. The vial recess 27 and the swab recess 28 are integrated in the housing 3 and respectively and substantially match the size and form of the vial 19 and the swab 29. The vial 19 and the swab 29 may be taken out of their holders to be used during a test. The vial recess 27 and/or the swab recess 28 may specifically be dimensioned such that the vial 19 and the swab 29 do not exceed the surface of the housing 3. As can be seen in this figure, the cap 19′ of the vial 19 and the upper surface of the swab 29 are flush with the upper surface of the housing 3. This has the advantage that multiple test sets 1 may be stapled and shipping efficiency can therefore be optimized.

In the embodiment of FIG. 9g, the application recess 8 has a round shape as an example, however, the shape of the application recess 8 may be different, such as squared, oval or polygonal and the side walls extending towards the insert tray 4 may be rectangular or tapered. In general, tapered side walls provide a larger application recess 8 on the upper side such that a user can easily apply the sample liquid without spilling the liquid, wherein the liquid may flow down the walls towards the test strip 2 if the user does not exactly meet the lower opening which is closest to the test strip 2.

The advantage of providing a housing 3 that integrates holders 27, 28 for other elements of the test set 17 such as at least one vial 19 and/or at least one swab 29 is provided in that the structure of the test set 17 is compact and elements are less likely to be lost.

In general, the respective housing 3 of an embodiment described herein may be for single-use only. In that case, the housing 3 is already equipped with the test strip 2, as, for example, described in terms of one of the methods herein. Alternatively, at least the housing 3 as one component of the test set 17 may be re-used. In that case, the other elements, such as the test strip 2, the vial 19 and/or the swab 28 may be provided as single-use elements/consumables. A user may equip the housing 2 with the consumables himself or submit the housing to the provider who takes care of the cleaning and/or the equipment of the housing 3. Specifically, the housing 3 of FIG. 9g and FIG. 9h may be re-used, as it consists of a big piece and/or block of material. In order to reduce the mass of the housing, a recessed structure may be provided on the lower side of the housing 3 such that material can be saved, the housing 3 becomes light while at the same time maintaining the stability.

In all applicable embodiments of the test sets 17, the vial 19 may comprise an applicator, specifically an applicator provided at or on the cap 19′. The applicator may be provided thereon permanently or may be coupled to the vial 19 once it is opened. The applicator may be configured to safely transfer and/or dose the liquid onto the test strip 2. If the applicator is not permanently coupled and/or connected to the vial 19, the housing 3 may also provide a holder and/or a recess for receiving and/or storing the applicator. Alternatively, the liquid may be transferred from the vial 19 to the test strip 2 by using a pipette and/or syringe. The housing 3 may then provide a holder and/or a recess for receiving and/or storing the pipette and/or syringe.

In general, the application recess 8 may comprise an element of a coupling mechanism that couples with a counterpart, which is provided at a vial, a syringe or another container. This makes it easy to transfer the sample liquid from the container to the test strip 2 in a spill-free manner. The coupling mechanism may be based on a thread system, a bayonet coupling or another a piece that is fitted to its counter piece.

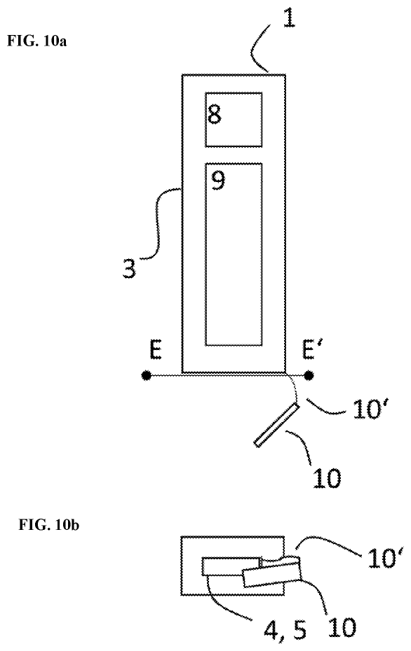

FIG. 10a is a schematic drawing showing a top view of a housing 3 with an insert plug 10 according to one embodiment. FIG. 10b is a schematic drawing showing a front view of the housing 3 of FIG. 10a along the line EE′. The insert plug 10 is connected with the housing 3 by means of a flexible bridge portion 10′. In this way, the insert plug 10 does not get lost when it is removed from the insert hole 5. The housing 3 may be formed as a single piece with the flexible bridge portion 10′ and the insert plug 10. Alternatively, the flexible bridge portion 10′ may be coupled to the housing 3 and formed as a single piece. The flexible bridge portion 10′ may be formed as one piece together with the insert plug 10 or alone being coupled to the insert plug 10. The flexible bridge portion 10′ may, for example, correspond to a flexible band or a string. In an alternative embodiment, these features apply to a cap 30 instead of an insert plug 10.

FIG. 11a is a perspective side view of a schematic drawing of the IVA test unit 1 having an insert tray 4 positioned lower than the (lowest position 5c of the) insert hole 5, while the insert hole 5 is tapered from the outer side 5a towards the inner side 5b, to reduce the risk of losing the test strip 2 via the insert hole 5 according to one embodiment. FIG. 11b is a front view of a schematic drawing of the IVA test unit 1 of FIG. 11a.

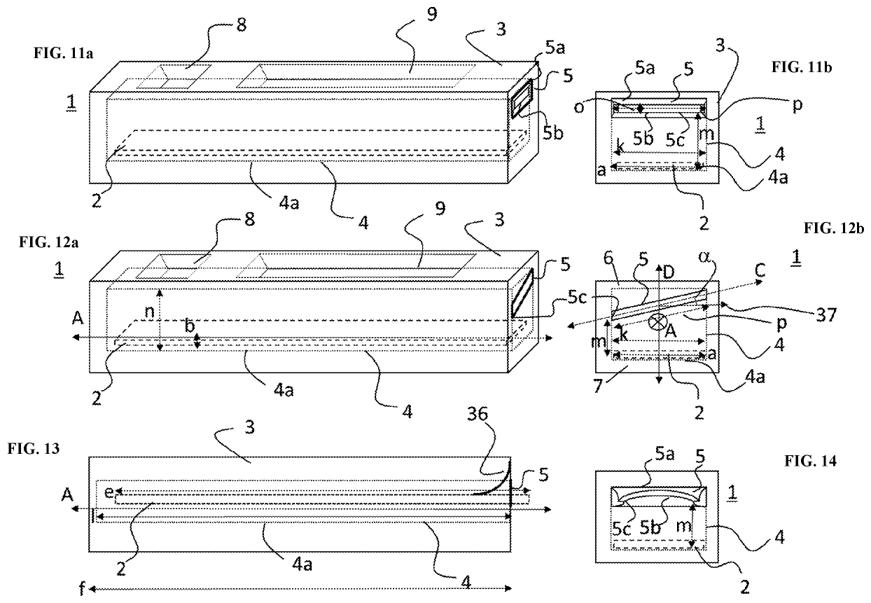

FIG. 12a is a perspective side view of a schematic drawing of the IVA test unit 1 having an insert hole 5 tilted with respect to a plane perpendicular to the height axis D of the housing 3 (i.e., specifically a plane 37 being parallel to the upper and/or lower walls 6, 7 of the housing 3) to reduce the risk of losing the test strip 2 via the insert hole 5 according to one embodiment. FIG. 12b is a front view of a schematic drawing of the IVA test unit 1 of FIG. 12a.

FIG. 13 is a side view of a schematic drawing of the IVA test unit 1 having a flap 36 in front of the insert hole 5 to reduce the risk of losing the test strip 2 via the insert hole 5 according to one embodiment.

FIG. 14 is a front view of a schematic drawing of the IVA test unit 1 having a curved inner side 5b of the insert hole 5, while the insert hole 5 is tapered (in a non-linear fashion) from the outer side 5a towards the inner side 5b, to reduce the risk of losing the test strip 2 via the insert hole 5 according to one embodiment.

In FIG. 11a-14, the insert tray 4 inside the housing 3 is indicated by a dotted line. The test strip 2 positioned inside the insert tray 4 is indicated by a dashed line. As can be seen in both figures, the outer side 5a of the insert hole 5 is tapered towards the inner side 5b of the insert hole 5.

In general, the insert tray 4 may in some cases be considered as the internal volume of the housing 3. The insert tray 4 may provide a volume for housing the test strip 2 that is, without limitation, approximately 1.1 to 20 times or more larger than the volume of the test strip 2 itself. Specifically, the height n of the insert tray 4 may be, without limitation, approximately 1.1 to 20 times or more larger than the height b of the test strip 2 (see FIG. 12a). The widthk of the insert tray 4 may be, without limitation, approximately 1.01 to 1.5 times larger than the width a of the test strip 2 (see FIGS. 11b and 12b). The length l of the insert tray 4 may be, without limitation, approximately 1.01 to 1.5 times larger than the length e of the test strip 2 (see FIG. 13). The insert tray 4 may have a lower surface 4a that corresponds approximately to the area (length×width) of the test strip 2 (or is slightly larger). By choosing a lower surface 4a area (length×width) of the insert tray 4 that is only slightly bigger than the area (length×width) of the test strip 2, the test strip may be well positioned inside the housing 3 without the test strip 2 having the freedom to shake and/or move around a lot in a plane parallel to the area of the insert tray 4. In other words, the insert tray 4 defines the position of the test strip 2 very well such that the test strip 2 is substantially fixed at least in a direction along the plane, i.e., the lower surface 4a of the insert tray 4.

The lower surface 4a of the insert tray 4 may lie in a plane that is defined by the length l and the width k of the insert tray 4 and that is considered as the upper surface of the second wall 7, i.e., the lower wall of the housing 3. The lower surface 4a of the insert tray 4 therefore faces the lower side of the upper wall 6 of the housing 3.

The lower surface 4a of the insert tray 4 may be in one plane with the lowest point of the insertion opening 5. Alternatively and as shown in FIG. 11a to FIG. 14, the lower surface 4a of the insert tray 4 is located deeper than the lowest point 5c of the insert hole 5 (see, for example, the according reference numerals in FIG. 11b, FIG. 12a, FIG. 12b and FIG. 14). The lower surface 4a of the insert tray 4 may hence be positioned lower than the insert hole 5. In other words, the insert tray 4 is set back and/or set lower into the lower wall 7 of the housing 3 with respect to the lowest point 5c of the insert hole 5. Specifically, the lower wall 7 of the housing 3 which defines the lower surface 4a of the insert tray may be recessed inside the interior volume to receive the test strip 2 in a position that is positioned lower than the lowest point of the insert hole 5. In that way, the test strip 2 may be inserted into the insert tray 4 via the insert hole 5 and the test strip 2 may then fall down onto the lower surface 4a of the insert tray 4 driven by gravity. Without limitation, the recess, which corresponds to the depth and/or the distance from lowest point 5c of insert hole 5 to the lower surface 4a of the insert tray 4 may be approximately 0.1 cm to 1 cm, specifically approximately 0.2 cm to 0.7 cm and preferably approximately 0.3 cm to 0.5 cm. The distance between the lowest point 5c of insert hole 5 to the lower surface 4a of the insert tray 4 may be approximately 1.5 to 20 or more times larger than the test strip 2. The distance between the lowest point 5c of insert hole 5 to the lower surface 4a of the insert tray 4 may be approximately 1.5 to 20 or more times larger than the slit size o on the inner side 5b of the insert hole 5. This may help securing the test strip 2 inside the insert tray 4 such that it does not fall out easily.

As shown in FIGS. 11a, 11b and FIG. 14, the insert hole 5 is tapered from the outer side 5a towards the inner side 5b. This feature is compatible with other embodiments described herein, such as the embodiments of FIG. 2, 5-10b, 12a, 12b, and 13. The insert hole 5 comprises a slit having a slit size “o” (already mentioned above as height of insert hole). The slit size “o” may in most cases be measured in a perpendicular direction to the length axis C of the insert hole 5. The slit size “o” may be, without limitation, approximately 1.01 to 2 times larger than the height “b” of the test strip 2 such that the test strip 2 is only slightly thinner than the slit size “o.” The width “p” of the insert hole 5, measured in most cases along the direction of the length axis C, may substantially correspond to the width “k” of the insert tray 4 and/or the width “a” of the test strip 2. The width “p” of the insert hole 5 may be slightly smaller or bigger than the width “k” of the insert tray 4 and/or slightly bigger than the width “a” of the test strip 2. The width “p” of the insert hole 5 may be, without limitation, approximately 1.01 to 2 times larger than the width “a” of the test strip 2 such that the test strip 2 is only slightly smaller in its width than the insert hole 5. In other words, the insert hole 5, which may be tapered towards the insert tray 4, may be only slightly larger than the test strip 2 in its cross section such that it is not easy for the test strip 2 to meet the exact position of the insert hole 5 from the inside of the housing 3 when it is fully inserted, thereby reducing the risk of falling out. The insert hole 4, i.e., a tight slit, may specifically be only slightly larger than the test strip 2 on the inner side 5b of the insert hole 5, i.e., the side that is closest to the insert tray 4. Without limitation, the cross section area of the insert hole 5 may be larger than the cross section area of the test strip 2 by the factor of approximately 1.05 to 5, specifically by the factor of approximately 1.1 to 2 and preferably by the factor of approximately 1.2 to 1.7. If the insert hole 5 is tapered towards the insert tray 4, it allows an easy insertion of the test strip 2 and the insert hole 5 guides the test strip 2 with its tapered form towards the insert tray 4.

It is advantageous to provide a slightly broader and/or higher insert hole 5 and/or insert tray 4 with respect to the size of the test strip 2 to make insertion of the test strip 2 into the insert tray 4 possible. As already described above, the insert tray 4 may, without limitation, have a tapered and/or conically shaped insert hole 5, which makes it even easier and/or more reliable to insert the test strip 2 while the risk is reduced that the test strip 2 falls out of the insert tray 4 once the test strip 2 is fully inserted into the insert tray 4. This may be advantageous specifically if a user manually performs the insertion. It is also advantageous if a robot or machine performs the insertion. Alternatively, in some embodiments, it is not a requirement that the insert tray 4 and/or the insert hole 5 is tapered and/or conically shaped.

As shown in FIGS. 12a and 12b, the insert hole 5, which may substantially correspond to a slit, may be tilted by an angle α with respect to a plane that is parallel to the substantially parallel (horizontal) planes defined by the upper and the lower walls 6, 7 of the housing 3. Without limitation, the slit axis of the insert hole may be tilted with respect to the plane that is parallel to the substantially parallel (horizontal) planes defined by the upper and the lower walls 6, 7 of the housing by an angle α of approximately 3° to 45°, specifically by approximately 5° to 30°, and preferably by approximately 7° to 15°. As a result, the angle between the height axis of the housing (which is perpendicular to the substantially parallel and horizontal planes defined by the upper and the lower walls of the housing) and the slit axis C of the insert hole 5 may be slightly off 90° (90°+/−approximately 3° to 45°, specifically by approximately 90°+/−5° to 30°, and preferably by approximately 90°+/−7° to 15°). The test strip 2 and/or the housing 3 may therefore be slightly tilted with respect to each other (and/or with respect to the horizontal plane) when inserting the test strip 2 into the insert tray 4. A tilted insert hole 5 allows securing the test strip 2 inside the insert tray 4 in a simple way as the probability of the test strip 2 falling out of the insert tray 4 via the tilted insert hole 5 is very low since there is no surface that supports the test strip 2 in that angle α and therefore, it does not easily fall out.

As shown in FIG. 14, the insert hole 5 may be (slightly) curved and inserting the test strip 2 may therefore require a (slight) bending of the test strip 2 to meet the shape of the opening of the insert hole 5. The bending may be performed by an additional support that may be placed above and/or underneath the test strip 2 and that forces the test strip 2 into the required shape to be inserted via the curved insert hole 5. Alternatively or in addition, the insert hole 5 may be tapered from the outer side 5a towards the inner side 5b (possibly from an uncurved slit on the outside) towards a curved slit on the inside and in such a way that the test strip 2 is forced into the (slightly) bent and/or curved shape by the insertion process, specifically by a pushing movement of the test strip 2 into the insert tray. As most test strips 2 are resilient in shape and turn back into their flat shape once being inserted, they are safe from falling out of the insert tray 4 as they do not spontaneously adopt the curved shape.

As shown in FIG. 13, the insert hole 5 and/or the housing 3 may comprise a flexible lip 36 on the inside of the insert tray 4 which bends towards the insert tray 4 when inserting the test strip 2 and covers or even seals the insert hole 5 from inside the insert tray 4. The flexible lip 36 corresponds to a valve in its function and is configured to let pass the test strip 2 only in one direction, namely the insertion direction from the outside towards the inside of the housing 3/insert tray 4. The test strip 2 is then secured inside the insert tray 4 and does not fall out via the insert hole 5 since the flexible lip 36 is configured to only bend in one direction.

Therefore, resuming the above, the IVA test unit 1 may comprise at least one of the following features to reduce the risk of the test strip 2 falling out from the insert tray 4:
a lower surface 4a of the insert tray 4 (i.e., an (upper) surface of the above described second (lower) wall 7 on the inside of the housing 3 adjacent the (lower) surface of the first wall 6 on the inside of the housing 3) may be positioned lower than the lowest point 5c of the inner side 5b of the insert hole 5, specifically the lower surface 4a of the insert tray 4 is approximately 0.1 cm to 1 cm, specifically approximately 0.2 cm to 0.7 cm and preferably approximately 0.3 cm to 0.5 cm deep with respect to the lowest point 5c of the inner side 5b of the insert hole 5 (see FIGS. 11a, 11b, 12a, 12b, 13 and 14).
the insert hole 5 may be tapered from the outer side 5a of the insert hole 5 towards the inner side 5b of the insert hole 5, specifically such that the cross section area defined by the outer side 5a of the insert hole 5 is larger than the cross section area defined by the inner side 5b of the insert hole 5 and the side walls of the insert hole taper from the outer side 5a of the insert hole 5 to the inner side 5b of the insert hole 5 (see FIGS. 11a, 11b and 14).
the insert hole 5, specifically the inner side 5b of the insert hole 5 may be tilted by the angle α with respect to a plane 37 parallel to the first and the second walls 6, 7 of the housing 3, specifically the insert hole 5 may define a slit with a slit axis C, i.e., the length axis of the insert hole 5 specifically on the inner side 5b, and the slit axis C may be tilted with respect to the plane 37 parallel to the first and the second walls 6, 7 of the housing 3 and form an angle with the height axis D of the insert hole 5 that is off 90°, namely 90°+/−α (see FIGS. 12a, 12b).
the inner side 5b of the insert hole 5 may be formed in a bent shape and/or may be configured to shape/bent the test strip 2 when inserting the test strip 2 into the insert tray 4, specifically the outer side 5a of the insert hole 5 may be tapered towards the inner side 5b of the insert hole 5 such that the test strip 2 is brought further and further into a bent shape that fits the inner side 5b shape of the insert hole 5 upon inserting the test strip 2 (see FIG. 14).
the IVA test unit 1 may comprise a flexible and/or resilient flap 36 that is configured to cover the insert hole 5 from the inner side 5b of the insert hole 5, to bend inwards when inserting the test strip 2 and flip back and cover the insert hole 5 when the test strip 2 is fully inserted and prevent the test strip 2 from falling out via the insert hole 5 (see FIG. 13).

All these features may be realized in a relatively simple way by means of the said manufacturing methods (injection molding and/or additive manufacturing) and therefore effectively help securing the test strip 2 inside the housing 3 and/or reducing the risk that the test strip 2 falls out.

In general, it is an advantage to provide a housing 3 with an insert tray 4 that allows to provide a close contact between the test strip 2 and the application recess 8. In more detail, it is advantageous for the flow of liquids applied via the application recess 8 that the walls around the application recess 8 are in close contact with the test strip 2, specifically with the sample pad of the test strip 2. The flow of the liquid, which is applied, may thereby be guided by the application recess towards the sample pad of the test strip 2.

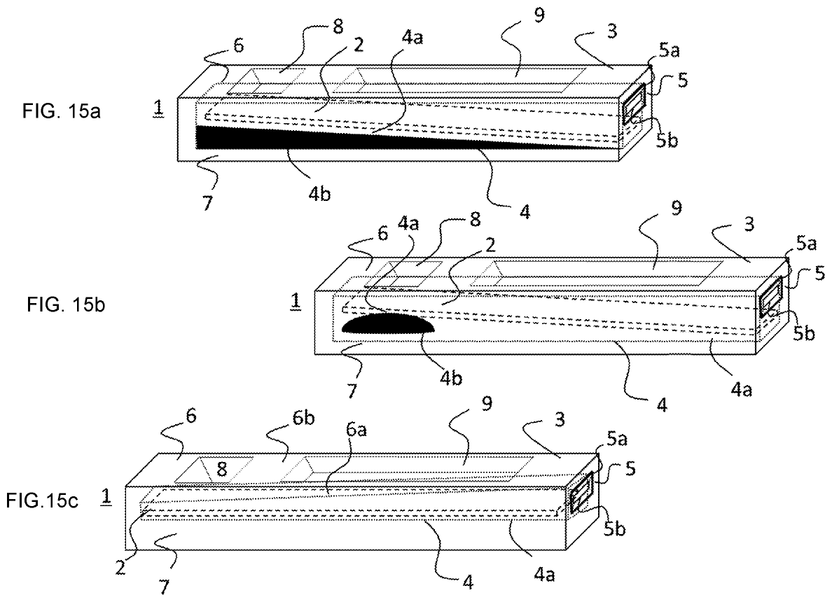

FIG. 15a is a schematic drawing of the IVA test unit 1 in a perspective side view having an insert tray 4 positioned lower than the insert hole 5 and the lower surface 4a of the insert tray is tilted with respect to the upper wall 6 of the housing 3 according to one embodiment. The upper and lower surfaces of the upper wall 6 are parallel to each other and lie in horizontal planes when the housing 3 is positioned in the proper upright orientation for correct usage. The tilted lower surface 4a of the insert tray 4 supports the test strip 2 such that the back portion of the test strip 2 is pressed against the upper wall 6 of the housing 3 and a tight contact between the wall portion around the application recess 8 and the test strip 2 can be established. The portion of the lower wall 7 that forms and/or defines the tilted lower surface 4a may be considered a support structure for the test strip 2 of the housing 3 and may be formed as a single piece with the entire housing 3. The angle between the tilted surface 4a and a plane, which is parallel to the upper wall, may range between approximately 1° and 20°, specifically between approximately 3° and 15°.

FIG. 15b is a schematic drawing of the IVA test unit 1 in a perspective side view having an insert tray 4 positioned lower than the insert hole 5 and on the lower surface 4a of the insert tray 4 a support structure 4b in the form of a half dome is provided according to one embodiment. The support structure is configured to press the back portion of the test strip 2 against the upper wall 6 of the housing 3 and a tight contact between the wall portion around the application recess 8 and the test strip 2 can be established. The support structure 4b of this embodiment may also be formed as a single piece with the entire housing 3.

FIG. 15c is a schematic drawing of the IVA test unit 1 in a perspective side view having an insert tray 4 positioned lower than the insert hole 5 and the lower surface 6a of the upper wall 6, i.e., the upper surface 6a of the insert tray 4, is tilted with respect to the upper surface 4a of lower wall 7 of the housing 3, i.e., the lower surface 4a of the insert tray 4. Further, the upper surface 6a of the insert tray 4 is tilted with respect to the upper surface 6b of the upper wall 6 of the housing 3. This configuration also allows establishing a tight contact between the test strip 2 and the upper wall 6, specifically the lower surface 6a of the upper wall 6 in the region of the application recess 8. The angle between the tilted lower surface 6a of the upper wall 6 and a plane, which is parallel to the upper surface 6b of the upper wall, may range between approximately 1° and 20°, specifically between approximately 3° and 15°. The depth of the application recess 8 corresponds to the thickness of the upper wall 6 and may therefore vary due to the varying thickness of the upper wall 6 in this embodiment.

In the following, some of the potential advantages of this disclosure are resumed again. A single-piece housing may increase the torsional stiffness and the value of the housing. No pick-and-place processes for the production of the housing is required. The test strip may be inserted into the insertion test disposable by means of a lifting movement and a bonding is not required. The “interior design” of the housing may prevent an “overflowing” “uncontrolled” flooding of the test strip. In one embodiment, the housing may comprise a material that is impermeable to water vapor and therefore, a pouch may not be required for packing the IVA test unit. Alternatively or in addition, the housing may be sprayed with a water vapor barrier, lacquered or coated by dipping into a bath of a water vapor-impermeable material. In that case, the openings of the housing, such as the insertion opening, the application recess (considered an insertion opening) and a recess in the control area (considered as a read-out window) may be covered by a foil and/or a film that is impermeable to water vapor to seal the inside of the housing. Such a foil may be bonded to the housing and may be stripped off the housing before usage of the IVA test unit. One individual piece of foil may be provided for each opening. Alternatively, one piece of foil may be provided for at least two openings of the housing. In this case, it may not be required to provide a pouch, such as an aluminum-PE bag.

In the following, some concepts, examples and embodiments, which may resemble or deviate from the above described embodiments, are resumed:

The (IVA) test strip may also be referred to insert test as the test strip is inserted into the housing. In general, the test strip, which can be used together with this disclosure or any embodiment thereof, may differ from the shown test strip. For example, the test strip may not have a substrate or may only have a membrane and a sample collection pad but no sample and conjugate pads if the reaction is performed outside of the test strip for example.

### Insert Test (Without Desiccant Properties)

The housings (in some case also referred to insert disposables) are filled into the plant as “loose bulk.” In a drum, these housings are fed into a system with the insert hole to the front and guided by means of a conveyor belt to the insertion of the test strip. By means of a guillotine cut, for example, the test strips are cut, separated and/or snipped out of the laminate roll.

### Insert Test With Desiccant Properties (Which May Be Integrated in the Housing in One Embodiment)

The finished insert test is sealed in a material that is impermeable to water vapor (e.g., aluminum-plastic composite, or HDPE etc.). In order to bind the moisture, a desiccant (usually a desiccant bag) is inserted into the packaging. A desiccant bag may therefore be added to and sealed in the packaging. The insert test can be made of a plastic with desiccant properties. This has the effect that no desiccant needs to be added as a separate element and therefore, the bag can be made smaller and/or more compact.

### Insert Test With a Clip-on Cap With Desiccant Properties

In this special embodiment, the housing has no desiccant properties. By attaching an additional cap (attachable and provided with desiccant properties), similar or the same advantages as for the housing with desiccant properties can be achieved. The advantages of this embodiment over an insert test with a housing that has desiccant properties:
The test strip insertion opening of the Insert-Test-Disposable can be closed
The test strip is no longer visible through the insertion opening and does not fall out
The plastic material of the housing can be made from an inexpensive plastic materials.

### Insert Test With a Functionalized Disposable Surface (e.g., Water Vapor Impermeable Coating)

In this special embodiment, there is the option of the insert test being made from an water vapor impermeable injection molded plastic and/or being functionalized with a water vapor impermeable coating, specifically being functionalized with a water vapor impermeable coating. Finally, the insert test can be glued or sealed, e.g., with an aluminum foil.

In the following some general statements are made. When liquids and/or liquid samples are applied to a sample pad of an LFA-core strip, lateral flow through the strip material may result from capillary forces, surface tension, cohesion, adhesion, wicking. In general, embodiments that utilize lateral flow may require substantial amounts of a liquid sample for sufficient contacting of the sample with a devices test area. Moreover, some embodiments overcome the use of lateral flow by having a test pad designed to absorb the liquid sample without requiring surface tension, cohesion, adhesion, wicking, and/or capillary action to contact the liquid sample with the test area. Such embodiments are particularly suited for use when the volume of a liquid sample is small. Such cases include, without limitations, instances when the liquid sample corresponds to or includes a capillary blood sample.

The insert hole 5 may in general be understood as an insert opening and/or an insert slit.

The terms “upper” and “lower” as used herein refer to the presented figures, particularly the figures, which show cross sections or the housing 3 where the upper surfaces are shown as upper surfaces in the real sense and where the lower surfaces are shown as lower surfaces in the real sense.

Modifications and variations of the disclosed aspects are also certainly possible in light of the above description. It is therefore to be understood, that within the scope of the appended claims, this disclosure may be practiced otherwise than as specifically devised in the above examples.

Particularly, it is to be understood that at least some of the drawings or parts are only schematic and provided as way of example only. Also the relationship between elements may be other than the one shown, whereas parts not relevant for the purpose of this disclosure have been omitted.

Also, reference throughout the preceding specification to “one aspect,” “an aspect,” “one example” or “an example,” “one embodiment” or “an embodiment” means that a particular feature, structure or characteristic described in connection with the aspect or example or embodiment is included in at least one aspect, example or embodiment. Thus, appearances of the phrases “in one aspect,” “in one aspect,” “one example” or “an example,” “one embodiment” or “an embodiment,” in various places throughout this specification are not necessarily all referring to the same aspect or example or embodiment.

Furthermore, the particular features, structures, or characteristics may be combined in any suitable combinations and/or sub-combinations in one or more aspects or examples or embodiments.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF REFERENCE NUMERALS

1 IVA test unit, specifically lateral flow assay (LFA) test unit
2 test strip, specifically an LFA-core strip
3 housing
4 insert tray
4a lower surface of the insert tray
4b support structure in insert tray
5 insert hole
5a outer side of insert hole
5b inner side of insert hole
5c lowest position of inner side of insert hole
6 first wall
6a lower surface of first wall/upper surface of the insert tray
6b upper surface of first wall
7 second wall
8 application recess
9 control area
10 insert plug
10′ flexible bridge portion to connect insert plug with housing
11 sample pad
12 conjugate pad
13 membrane
14 absorption pad
15 substrate of test strip
16 cutter
17 test set/test kit
18 band comprising a plurality of LFA-core strips
19 vial for housing a liquid
19′ vial cap
20 plurality of LFA-core strips
21 test line
22 control line
23 sample liquid
24 transparent cover plate
25 tip of a needle or a pipette
26 packaging foil
27 vial holder/recess
28 swab holder/recess
29 swab
30 cap
31 bridge for vial holder
32 bridge for swab holder
33 flexible porous head piece of swab
34 stick piece of swab
35 packaging for swab, specifically head piece
36 flap
37 plane parallel to the first and the second walls of the housing
38 coupling mechanism to couple a cap to a housing
100 method of producing an IVA test unit
110 providing a single-piece housing
110a producing the housing as a single piece by injection molding
110b producing the housing as a single piece by additive manufacturing
120 placing the LFA-core strip in the housing
121 providing a band
122 transporting the band
123 cutting the band
124 aligning the housing
125 inserting the LFA-core strip into the insert tray
130 plugging an insert plug into the insert hole
140 sealing the IVA test unit by means of a cover plate
200 prior art LFA-set
201 housing made of two pieces
201a upper piece of the housing
201b lower piece of the housing
202 LFA-core strip
203 packaging
204 drying agent
A longitudinal housing axis
B longitudinal band axis
C length axis of the insert hole/slit
D height axis of the housing
α angle between slit axis of the insert hole and height axis of the housing
a width of test strip
b height of test strip
c width of housing
d height of housing
e length of test strip
f length of housing
g width of application recess
h height of application recess
i height of control area
j width of control area
k width of the insert tray
l length of the insert tray
m depth from lowest point of insert hole to lower surface of insert tray
n height of the insert tray
o height of insert hole/slit size on the inner side of the insert hole
p width of the insert hole
AA′ cutline
BB′ cutline
CC′ cutline
DD′ cutline
EE′ Cutline

What is claimed is:

1. An IVA test unit, comprising:
a test strip for detecting an analyte, wherein the test strip comprises at least an application function, a test reaction function and a test detection function; and
a housing formed as one single piece and configured for storing the test strip, wherein the housing comprises an insert tray and an insert hole configured for inserting the test strip into the insert tray.

2. The IVA test unit of claim 1, wherein the housing comprises a first wall and a second wall adjacent the first wall, and wherein the first wall and the second wall define the insert tray between the first wall and the second wall.

3. The IVA test unit of claim 1, wherein the housing comprises:
an application recess configured for allowing the application of a sample liquid onto the test strip; and/or
a control area configured for reading out a test result from the test strip.

4. The IVA test unit of claim 1, further comprising one of the following closure elements:
an insert plug configured to be plugged into the insert hole to close the insert hole and/or secure the test strip from falling out of the insert tray;
a cap configured to be clipped to the housing over the insert hole.

5. The IVA test unit of claim 3, further comprising one of the following closure elements:
an insert plug configured to be plugged into the insert hole to close the insert hole and/or secure the test strip from falling out of the insert tray;
a cap configured to be clipped to the housing over the insert hole.

6. The IVA test unit of claim 3, further comprising at least one transparent cover plate configured to cover and/or close the control area and/or the application recess.

7. The IVA test unit of claim 5, further comprising at least one transparent cover plate configured to cover and/or close the control area and/or the application recess.

8. The IVA test unit of claim 7, further comprising at least one of the following:
a drying agent as a compound of the housing, the closure element and/or the cover plate;
a drying agent as an element placed in at least one chamber and/or recess of the housing, the insert plug and/or the cover plate for storing the drying agent.

9. The IVA test unit of claim 7, wherein the IVA test unit comprises at least one of the following:
a water-vapor-impermeable material which is comprised as an integral material of the housing, the closure element and/or the cover plate;
a water-vapor-impermeable upper layer sprayed and/or coated onto the housing, the closure element and/or the cover plate;
a water-vapor-impermeable film, preferably wherein the a water-vapor-impermeable film encloses the entire IVA test unit.

10. The IVA test unit of claim 8, wherein the IVA test unit comprises at least one of the following:
a water-vapor-impermeable material which is comprised as an integral material of the housing, the closure element and/or the cover plate;
a water-vapor-impermeable upper layer sprayed and/or coated onto the housing, the closure element and/or the cover plate;
a water-vapor-impermeable film, preferably wherein the a water-vapor-impermeable film encloses the entire IVA test unit.

11. The IVA test unit of claim 1, wherein the housing comprises a fixing mechanism configured to fix the position of the test strip in the insert tray, optionally wherein the fixing mechanism comprises at least one of:
a hook as an element of the housing and a recess configured as an element of the test strip configured to snap into the hook;
a spring element configured to fix the test strip by means of a spring force;
an element of the housing configured to fix the test strip by means of a friction force.

12. The IVA test unit of claim 11, wherein the housing comprises a fixing mechanism configured to fix the position of the test strip in the insert tray, optionally wherein the fixing mechanism comprises at least one of:
a hook as an element of the housing and a recess as an element of the test strip configured to snap into the hook;
a spring element which is configured to fix the test strip by means of a spring force;
an element of the housing configured to fix the test strip by means of a friction force.

13. A method of producing an IVA test unit comprising the following steps:
providing a single-piece housing for storing a test strip, wherein the housing comprises an insert tray and an insert hole for inserting the test strip into the insert tray;
placing the test strip in the housing.

14. The method of claim 13, wherein the providing of the housing comprises producing the housing as a single piece by injection molding and/or by additive manufacturing.

15. The method of claim 13, wherein the placing of the test strip into the housing comprises:
providing a band with a longitudinal band axis, preferably from a roll, wherein the band comprises the test strip among a plurality of test strips along the longitudinal band axis;
transporting the band along the longitudinal band axis;
cutting perpendicular to the longitudinal band axis the band to separate the test strip from the plurality of test strips;
aligning the housing with a longitudinal housing axis parallel to or perpendicular to the longitudinal band axis and with the insert hole facing the test strip; and
inserting the test strip into the insert tray through the insert hole.

16. The method of claim 13, further comprising closing the insert hole by at least one of the following:
plugging an insert plug into the insert hole;
clipping a cap to the housing over the insert hole.

17. The method of claims 13, further comprising providing a drying agent as at least one of the following:
a compound of the housing, the insert plug and/or the cap;
an element placed in at least one chamber and/or recess of the housing, the insert plug and/or the cap.

18. The method of claim 13, further comprising at least one of the following:
spraying and/or coating a water-vapor-impermeable upper layer onto the housing;
enclosing the entire IVA test unit with a water-vapor-impermeable film.

19. The method of claim 13, further comprising fixing the position of the test strip in the insert tray using a fixing mechanism based on a snapping mechanism, a spring force and/or a friction force.

20. A test set, comprising:
the IVA test unit according to claim 1;
a swab for collecting a biological sample; and
a vial comprising a liquid for eluting the sample.
